(19) United States
(12) Patent Application Publication
(10) Pub. No.: US 2026/0240489 A1
(43) Pub. Date: Aug. 20, 2026

(54) Interdependent Human Behavior Detection and/or Classification using Active Acoustic Sensing

(71) Applicant: Google LLC, Mountain View, CA (US)

(72) Inventors: Jason Daniel Guss, Aliso Viejo, CA (US); Xiaoran Fan, Irvine, CA (US); Trausti Thormundsson, Irvine, CA (US)

(73) Assignee: Google LLC, Mountain View, CA (US)

(21) Appl. No.: 19/165,387

(22) PCT Filed: Mar. 15, 2024
(86) PCT No.: PCT/US2024/020269

§ 371 Date: Sep. 15, 2025

Related U.S. Application Data
(60) Provisional application No. 63/490,657, filed on Mar. 16, 2023.
(60) Provisional application No. 63/616,360, filed on Dec. 29, 2023.

(51) Int. Cl. A61B 5/00; A61B 5/024

(52) U.S. Cl. CPC A61B 5/4557; A61B 5/02438; A61B 5/4806; A61B 5/6803; A61B 5/6817

(57) ABSTRACT

Techniques and apparatuses are described for performing interdependent human behavior detection and/or classification using active acoustic sensing. With active acoustic sensing, multiple human behaviors can be detected and/or classified during a given time period. Interdependent human behavior detection and/or classification involves using the detection and/or classification of a first human behavior to assist with the detection and/or classification of a second human behavior. With interdependent human behavior detection and/or classification, active acoustic sensing can increase the accuracy and/or reliability of human behavior detection and/or classification compared to other single-behavior-based techniques. Furthermore, interdependent human behavior detection and/or classification can be performed using a single type of sensing modality (e.g., active acoustic sensing) and using a single sensor in some implementations. By relying on active acoustic sensing instead of other sensing modalities, it can be cheaper and/or easier to implement interdependent human behavior and/or classification techniques within a hearable.

### BACKGROUND

Technological advances in medicine and healthcare are making it possible for people to live longer, healthier lives. To further achieve this, individuals have become interested in tracking their personal health. Health monitoring can motivate an individual to realize a particular fitness goal by tracking incremental improvements in the performance of the body's functions. Additionally, the individual can monitor the impact of various chronic illnesses on their body. With active feedback through health monitoring, the individual can live an active and full life with many chronic illnesses and quickly recognize situations in which it is necessary to seek medical attention.

Some devices that support health monitoring, however, can be obtrusive, uncomfortable, and expensive. As such, people may choose to forego health monitoring if the device negatively impacts their movement, causes inconveniences while performing daily activities, or is unaffordable. It is therefore desirable for health-monitoring devices to be comfortable and affordable, as well as portable and reliable, to encourage more users to take advantage of these features.

### SUMMARY

Techniques and apparatuses are described that utilize active acoustic sensing for interdependent human behavior detection and/or classification. A hearable, such as an earbud, is capable of performing a novel physiological monitoring process termed herein audioplethysmography. Audioplethysmography is an active acoustic method capable of sensing subtle changes observable at a user's outer and middle ear. Instead of relying on other auxiliary sensors, such as optical or electrical sensors, audioplethysmography involves transmitting and receiving acoustic signals that at least partially propagate within a user's ear canal. To effectively perform audioplethysmography, the hearable should form at least a partial seal in or around the user's outer ear. This seal enables formation of an acoustic circuit, which includes the seal, the hearable, the ear canal, and an ear drum of the ear.

By transmitting and receiving acoustic signals, the hearable can recognize changes in the acoustic circuit to detect and/or classify one or more human behaviors. Example human behaviors include chewing, teeth clenching/grinding/tapping (bruxism), and/or sleeping. In example implementations, the detected and/or classified human behavior can control and/or change an operation of the hearable and/or a computing device that is coupled to the hearable.

With active acoustic sensing, multiple human behaviors can be detected and/or classified during a same time period. This enables the hearable to perform interdependent human behavior detection and/or classification, which involves using the detection and/or classification of a first human behavior to assist with (or enhance) the detection and/or classification of a second human behavior. With interdependent human behavior detection and/or classification, active acoustic sensing can increase the accuracy and/or reliability of human behavior detection and/or classification compared to other single-behavior detection and/or classification techniques. Furthermore, interdependent human behavior detection and/or classification can be performed using a single type of sensing modality (e.g., active acoustic sensing) and using a single sensor in some implementations. This differs from other human behavior techniques that may rely on multiple sensors (of a same type or of different types) to respectively detect multiple human behaviors. By relying on active acoustic sensing instead of other sensing modalities, it can be cheaper and/or easier to implement interdependent human behavior and/or classification techniques within the hearable. Some hearables can be configured to support active acoustic sensing without the need for additional hardware. As such, the size, cost, and power usage of the hearable can help make interdependent human behavior detection and/or classification accessible to a larger group of people and improve the user experience with hearables.

Aspects described below include a method for performing interdependent detection and/or classification of human behavior using active acoustic sensing. The method includes transmitting an acoustic transmit signal that propagates within at least a portion of an ear canal of a user. The method also includes receiving an acoustic receive signal, the acoustic receive signal representing a version of the acoustic transmit signal with one or more characteristics modified due to the propagation within the ear canal. The method additionally includes detecting a first human behavior based on the acoustic receive signal. The method further includes detecting a second human behavior based on the acoustic receive signal and the detected first human behavior, the second human behavior being different than the first human behavior. The method also includes controlling an operation of a device based on the detected second human behavior. The device may comprise at least one of a hearable or a computing device that is coupled to the hearable. This hearable may also be used for transmitting the acoustic transmit signal and/or for receiving the acoustic receive signal.

Aspects described below include a computer-readable storage medium comprising instructions that, responsive to execution by at least one processor, cause a device to perform any one of the methods described herein. In one example, the at least one processor may be part of the device of which an operation is controlled based on the detected human behavior or the at least one processor may be part of a hearable coupled to the device of which an operation is controlled based on the detected human behavior.

Aspects described below include a device with at least one transducer and at least one processor. The device is configured to perform, using the at least one transducer and the at least one processor, any one of the methods described herein. For example, the at least one transducer may be configured to transmit the acoustic transmit signal and/or to receive the acoustic receive signal.

Aspects described below include a system with means for performing interdependent human behavior detection and/or classification using active acoustic sensing.

### BRIEF DESCRIPTION OF DRAWINGS

Apparatuses and techniques for performing interdependent human behavior detection and/or classification using active acoustic sensing are described with reference to the following drawings. The same numbers are used throughout the drawings to reference like features and components:

FIG. 1-1 illustrates an example environment in which interdependent human behavior detection and/or classification using active acoustic sensing can be implemented;

FIG. 1-2 illustrates an example geometric change in an ear canal, which can be detected using active acoustic sensing;

FIG. 2-1 illustrates an example environment in which chewing detection can be performed using active acoustic sensing;

FIG. 2-2 illustrates example environments in which bruxism detection and/or classification can be performed using active acoustic sensing;

FIG. 2-3 illustrates various types of bruxism that can be detected and classified using active acoustic sensing;

FIG. 3-1 illustrates an example environment in which sleep detection and/or classification can be performed using active acoustic sensing;

FIG. 3-2 illustrates example biometrics that can be monitored for sleep detection and/or classification;

FIG. 4 illustrates an example implementation of a computing device;

FIG. 5 illustrates an example implementation of a hearable;

FIG. 6 illustrates example operations of two hearables;

FIG. 7 illustrates an example implementation of a hearable capable of performing interdependent human behavior detection and/or classification using active acoustic sensing;

FIG. 8 illustrates an example flow diagram for operating a hearable;

FIG. 9 illustrates an example scheme implemented by a calibration module of a hearable;

FIG. 10 illustrates an example implementation of a pre-processing module for performing aspects of active acoustic sensing;

FIG. 11 illustrates an example implementation of a measurement module for performing chewing detection, bruxism detection, and/or bruxism classification;

FIG. 12-1 illustrates an example implementation of a measurement module for performing sleep detection and/or sleep classification;

FIG. 12-2 illustrates an example implementation of a biometric measurement module;

FIG. 13 illustrates example audioplethysmography signals associated with chewing detection;

FIG. 14 illustrates first example audioplethysmography signals associated with bruxism detection and/or classification;

FIG. 15 illustrates second example audioplethysmography signals associated with bruxism detection and/or classification;

FIG. 16 illustrates third example audioplethysmography signals associated with bruxism detection and/or classification;

FIG. 17 illustrates fourth example audioplethysmography signals associated with bruxism detection and/or classification;

FIG. 18 illustrates example autocorrelations associated with biometric monitoring;

FIG. 19 illustrates an example audioplethysmography signal associated with biometric monitoring;

FIG. 20 illustrates example audioplethysmography signals associated with sleep classification;

FIG. 21 illustrates first example audioplethysmography signal associated with sleep detection and/or classification;

FIG. 22 illustrates second example audioplethysmography signals associated with sleep detection and/or classification;

FIG. 23 illustrates third example audioplethysmography signals associated with sleep detection and/or classification;

FIG. 24 illustrates an example method for detecting and/or classifying human behavior using active acoustic sensing;

FIG. 25 illustrates another example method for detecting and/or classifying human behavior using active acoustic sensing;

FIG. 26 illustrates an example method for detecting chewing using active acoustic sensing;

FIG. 27 illustrates an example method for detecting and/or classifying bruxism using active acoustic sensing;

FIG. 28 illustrates an example method for detecting and/or classifying sleep using active acoustic sensing;

FIG. 29 illustrates an example method for performing interdependent human behavior detection and/or classification using active acoustic sensing;

FIG. 30 illustrates another example method for performing interdependent human behavior detection and/or classification using active acoustic sensing; and

FIG. 31 illustrates an example computing system embodying, or in which techniques may be implemented that enable use of, interdependent human behavior detection and/or classification using active acoustic sensing.

### DETAILED DESCRIPTION

Technological advances in medicine and healthcare are making it possible for people to live longer, healthier lives. To further achieve this, it can be desirable to monitor and/or evaluate conscious and/or unconscious human behavior that impacts health of the human body. With active feedback through monitoring human behavior, an individual can make informed decisions to improve their health or seek further medical attention if necessary.

Some monitoring devices, however, can be obtrusive, uncomfortable, or socially awkward to wear. To detect bruxism, for instance, a user can adhere an ultrasonic sensor to a portion of their skin that is proximate to the jaw area. As it may be awkward and/or uncomfortable for the user to use this ultrasonic sensor, especially when they go out in public, the user may forego wearing this device during the day. This means that daytime occurrences of bruxism go undetected. The capabilities of some devices for monitoring bruxism may also be limited. Although a device may be able to detect the occurrence of bruxism, it may be unable to distinguish between different types of bruxism, for instance. Identifying the different types of bruxism can be valuable for evaluating the progression of bruxism-related issues and/or identifying solutions to prevent bruxism.

To monitor sleep, some devices are worn on the user's head, which can make it uncomfortable to sleep. To address this problem, other sleep-monitoring devices may be designed to be worn on the user's wrist or may be operated from a remote position. These devices, however, may not be as accurate in determining sleep quality.

Other monitoring devices may utilize auxiliary sensors, including optical or electronic sensors, that add additional weight, cost, complexity, and/or bulk. Still other devices may require constant recharging of a battery due to relatively high power usage. As such, people may choose to forego monitoring if the device negatively impacts their life. It is therefore desirable for human-behavior monitoring devices to be reliable, portable, efficient, and affordable to expand accessibility to more users.

Wireless technology has become prevalent in everyday life, making communication and data readily accessible to users. One type of wireless technology are wireless hearables, examples of which include wireless earbuds and wireless headphones. Wireless hearables have allowed users freedom of movement while listening to audio content from music, audio books, podcasts, and videos. With the prevalence of wireless hearables, there is a market for adding additional features to existing hearables utilizing current hardware (e.g., without introducing any new hardware).

Provided according to one or more preferred embodiments is a hearable, such as an earbud, that is capable of performing a novel physiological monitoring process termed herein audioplethysmography. Audioplethysmography is an active acoustic method capable of sensing subtle physiologically-related changes observable at a user's outer and middle ear. Instead of relying on other auxiliary sensors, such as optical or electrical sensors, audioplethysmography involves transmitting and receiving acoustic signals that at least partially propagate within a user's ear canal. To effectively perform audioplethysmography, the hearable should form at least a partial seal in or around the user's outer ear. Such a seal enables formation of an acoustic circuit, which includes the seal, the hearable, the ear canal, and an ear drum of the ear.

By transmitting and receiving acoustic signals, the hearable can recognize changes in the acoustic circuit to detect and/or classify one or more human behaviors. Example human behaviors include chewing, teeth clenching/grinding/tapping (bruxism), and/or sleeping. In example implementations, the detected and/or classified human behavior can control and/or change an operation of the hearable and/or a computing device that is coupled to the hearable.

With active acoustic sensing, multiple human behaviors can be detected and/or classified during a same time period. This enables the hearable to perform interdependent human behavior detection and/or classification, which involves using the detection and/or classification of a first human behavior to assist with (or enhance) the detection and/or classification of a second human behavior. With interdependent human behavior detection and/or classification, active acoustic sensing can increase the accuracy and/or reliability of human behavior detection and/or classification compared to other single-behavior detection and/or classification techniques. Furthermore, interdependent human behavior detection and/or classification can be performed using a single type of sensing modality (e.g., active acoustic sensing) and using a single sensor in some implementations. This differs from other human behavior techniques that may rely on different sensors (of a same type or of different types) to detect different human behaviors. By relying on active acoustic sensing instead of other sensing modalities, it can be cheaper and/or easier to implement interdependent human behavior and/or classification techniques within the hearable. In addition to being relatively unobtrusive, some hearables can be configured to support audioplethysmography without the need for additional hardware. As such, the size, cost, and power usage of the hearable can help make interdependent human behavior detection and/or classification accessible to a larger group of people and improve the user experience with hearables.

### Operating Environment

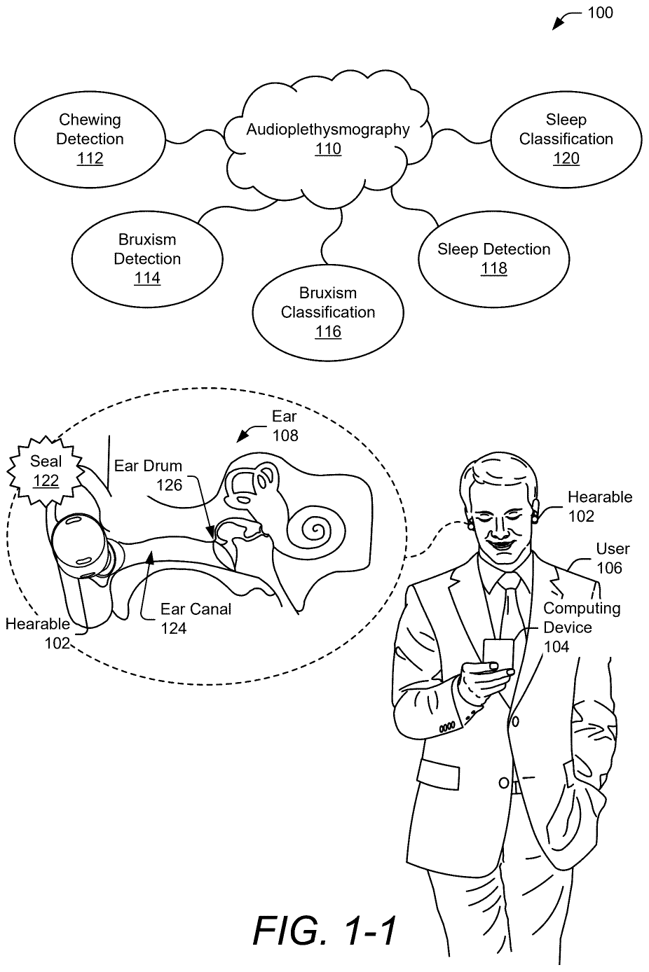

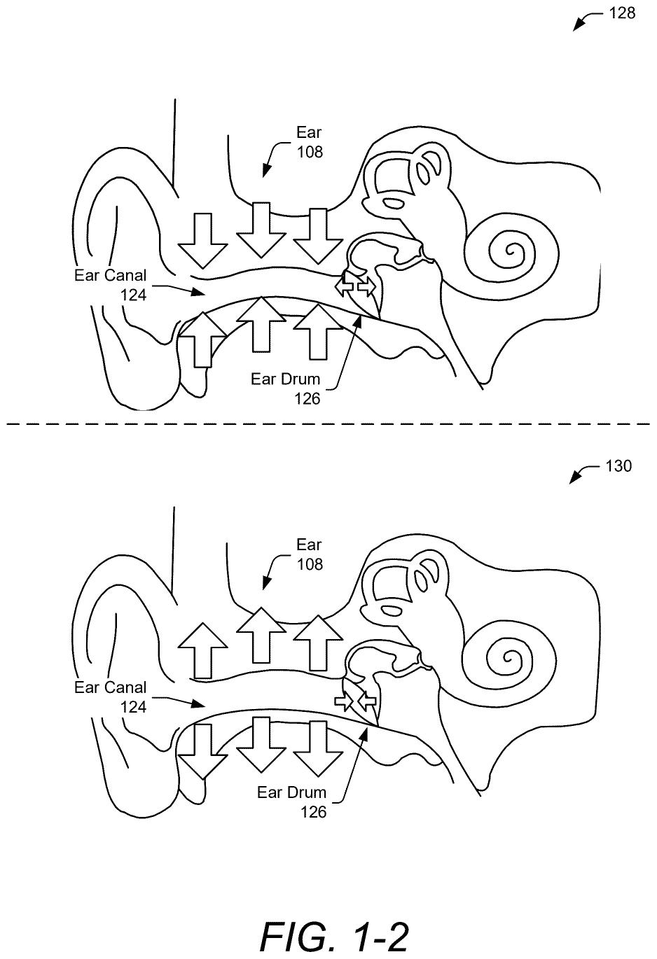

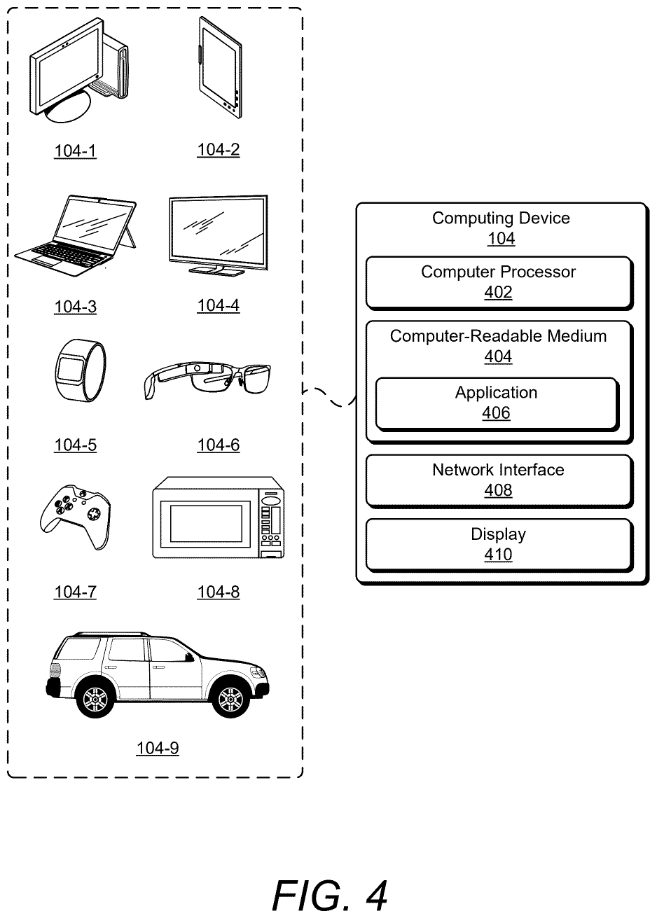

FIG. 1-1 is an illustration of an example environment 100 in which active acoustic sensing can be implemented. In the example environment 100, a hearable 102 is connected to a computing device 104 using a physical or wireless interface. The hearable 102 is a device that can play audible content provided by the computing device 104 and direct the audible content into a user 106's ear 108. In this example, the hearable 102 operates together with the computing device 104. In other examples, the hearable 102 can operate or be implemented as a stand-alone device. Although depicted as a smartphone, the computing device 104 can include other types of devices, including those described with respect to FIG. 4.

The hearable 102 is capable of performing audioplethysmography 110, which is an active acoustic method of sensing that occurs at the ear 108. The hearable 102 can perform this sensing without the use of other auxiliary sensors, such as an optical sensor or an electrical sensor. Through audioplethysmography 110, the hearable 102 can detect and/or classify various human behaviors, including chewing (or eating), bruxism, and sleep. In particular, the hearable 102 can use active acoustic sensing to perform chewing detection 112, bruxism detection 114, bruxism classification 116, sleep detection 118, sleep classification 120, or some combination thereof. In some implementations, the hearable 102 can also use active acoustic sensing to perform coughing detection.

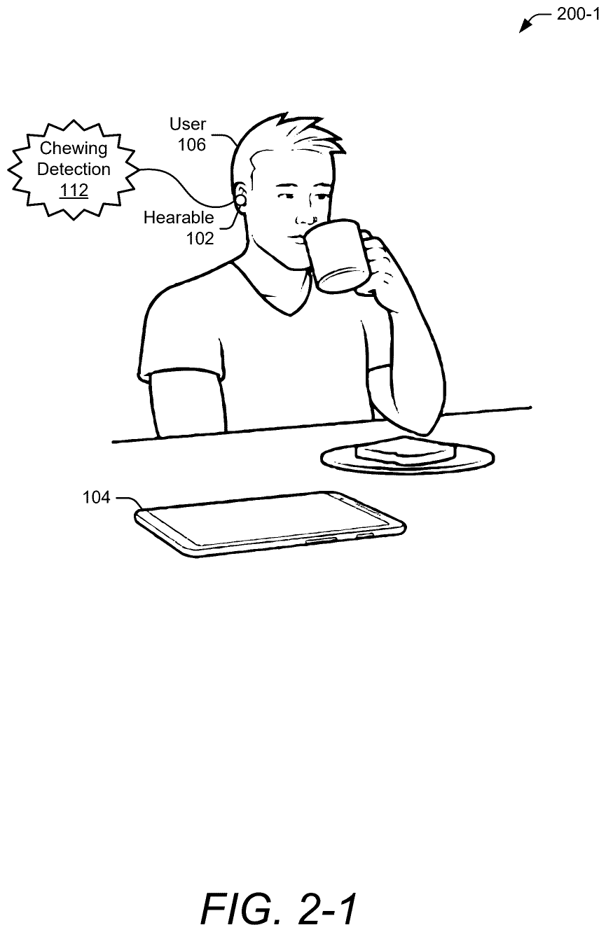

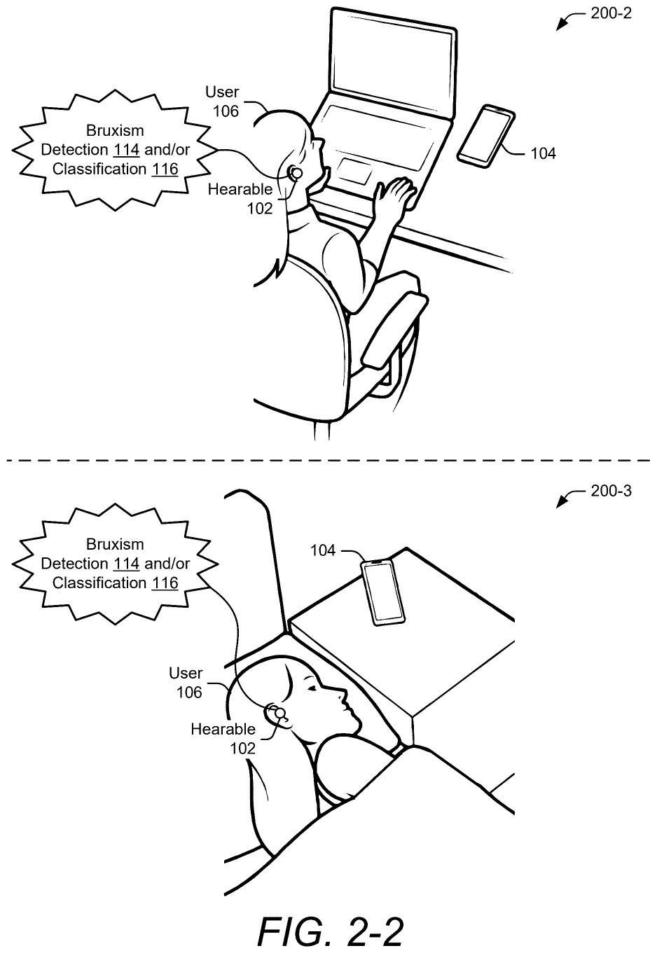

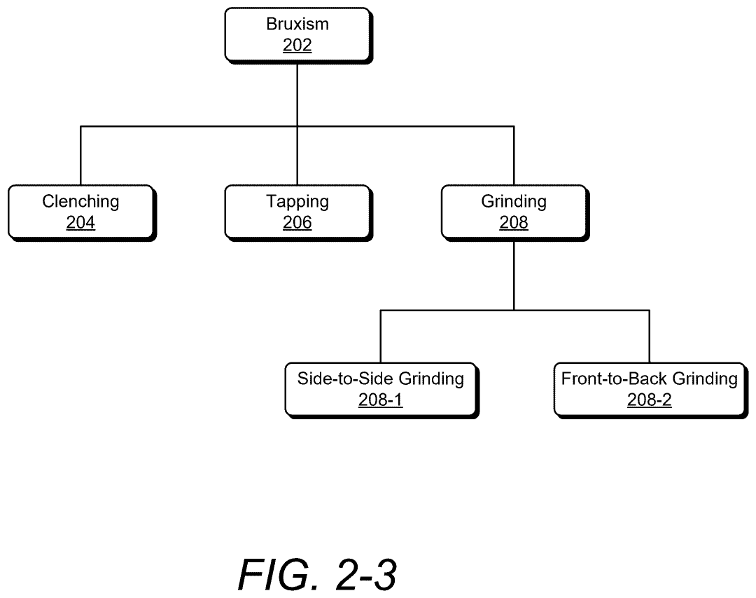

Chewing detection 112 detects or determines when the user 106 moves their jaw in a manner associated with chewing food. With chewing detection 112, a user 106's eating habit can be monitored and/or tracked, which can assist the user 106 in achieving dieting goals or changing their eating habit. Chewing detection 112 is further described with respect to FIG. 2-1.

Bruxism is a condition in which the user 106 grinds or clenches their teeth, usually in an unconscious manner. In severe cases, bruxism can cause excessive wear on teeth and jaw tenderness, which can result in teeth loss, an abnormal bite, or crooked teeth. It can also be an indication of sleep apnea or stress. Bruxism detection 114 can detect the occurrence (or absence) of bruxism. With bruxism detection 114, a severity of the user 106's bruxism can be monitored and/or tracked. This information can also be used to evaluate the user 106's stress levels and provide recommendations for decreasing stress. Bruxism detection 114 is further described with respect to FIG. 2-2.

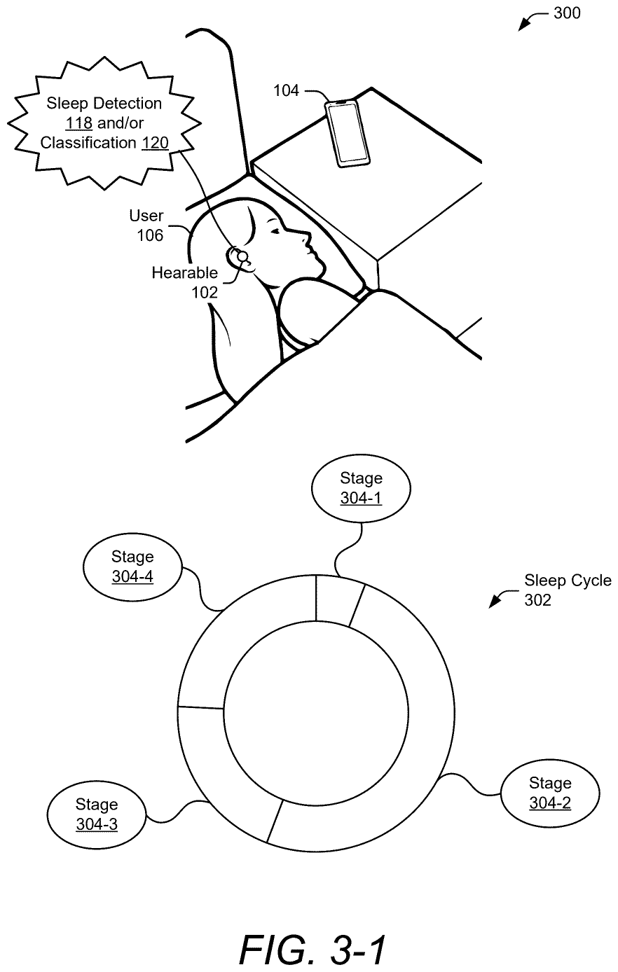

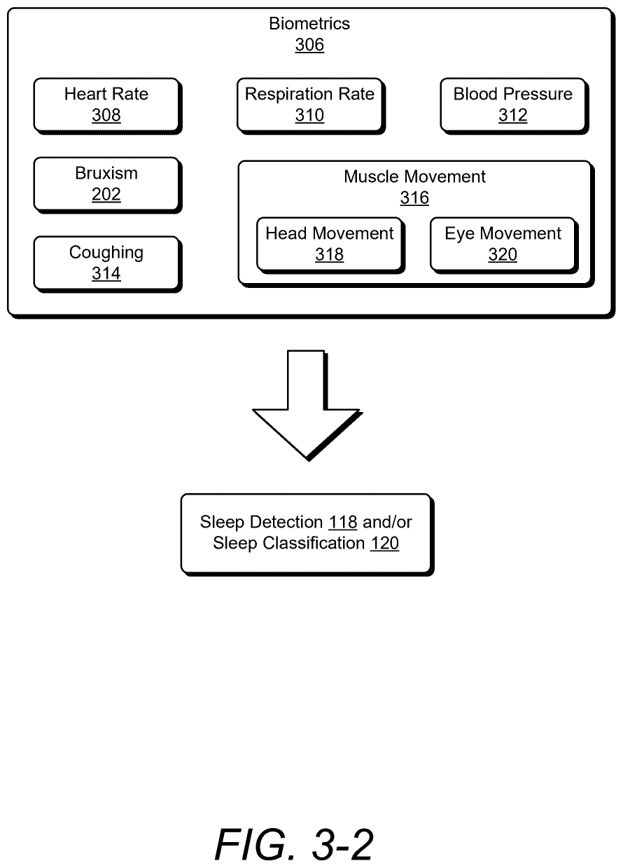

Bruxism classification 116 further identifies a manner in which bruxism presents itself. Different types of bruxism, for instance, can involve the user 106 clenching their teeth or grinding their teeth in a particular direction. By identifying the types of bruxism, bruxism classification 116 can be used to further enhance bruxism detection 114 by removing false positives associated with other human behaviors, such as chewing. Additionally or alternatively, bruxism classification 116 can be used to evaluate the progression of bruxism-related issues, such as temporomandibular disorder (TMD) and/or identify solutions to help prevent bruxism. Bruxism classification 116 is further described with respect to FIG. 2-3.

Using audioplethysmography 110 to perform chewing detection 112, bruxism detection 114, and/or bruxism classification 116 can provide better signal-to-noise performance compared to other types of sensors or components, such as a microphone or a voice accelerometer. This is because the signal generated using audioplethysmography 110 can have a significantly lower noise level compared to a signal generated using a microphone or a voice accelerometer. Additionally or alternatively, the signal generated using audioplethysmography 110 can have a significantly higher intensity in response to chewing and/or bruxism.

While these other sensors or components may be able to detect some types of bruxism, such as tapping, other types of bruxism, such as clenching and/or grinding, may be more challenging to detect and can be obscured by noise. These other types of bruxism can be more challenging for these other sensors or components to detect as they involve smaller movements and/or do not produce a significantly loud sound. In contrast, audioplethysmography 110 can readily detect these types of bruxism because audioplethysmography 110 does not rely on sound and instead detects changes in the geometric shape of the ear canal 124. Even small movements associated with certain types of bruxism can change the geometric shape of the ear canal 124 and cause a significant change in the amplitude and/or phase of an acoustic signal that is transmitted and received using audioplethysmography 110.

Sleep detection 118 can determine whether the user 106 is awake or asleep. With sleep detection 118, the user 106's sleep habits can be monitored and/or tracked, which can assist the user 106 in meeting sleep goals or changing their sleeping habit. Sleep classification 120 can further identify the various stages of sleep. With this information, sleep classification 120 can determine how long the user 106 spends in each sleep stage and/or evaluate the quality of the user 106's sleep. Sleep detection 118 and sleep classification 120 are further described with respect to FIGS. 3-1 and 3-2.

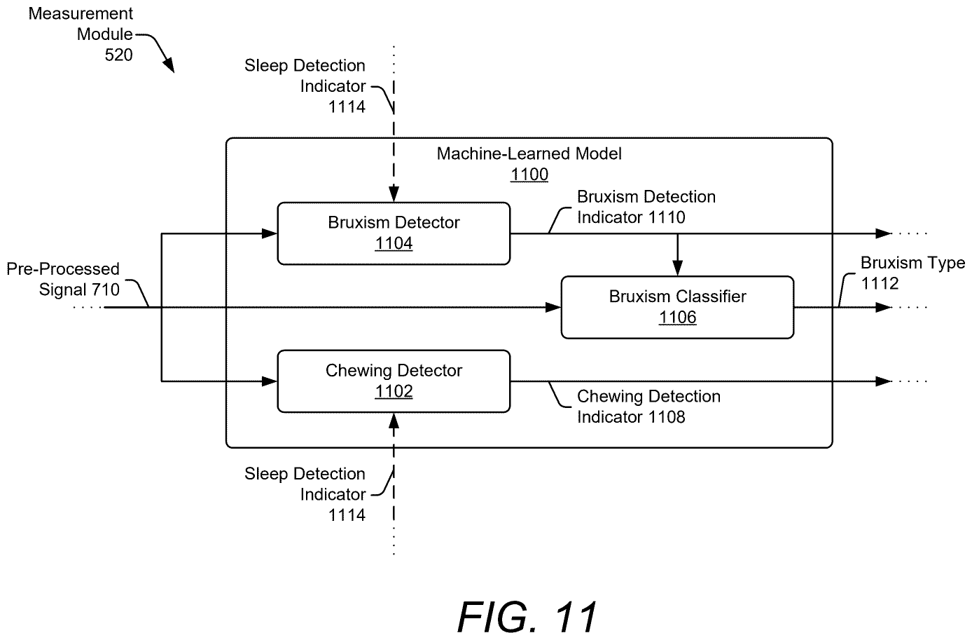

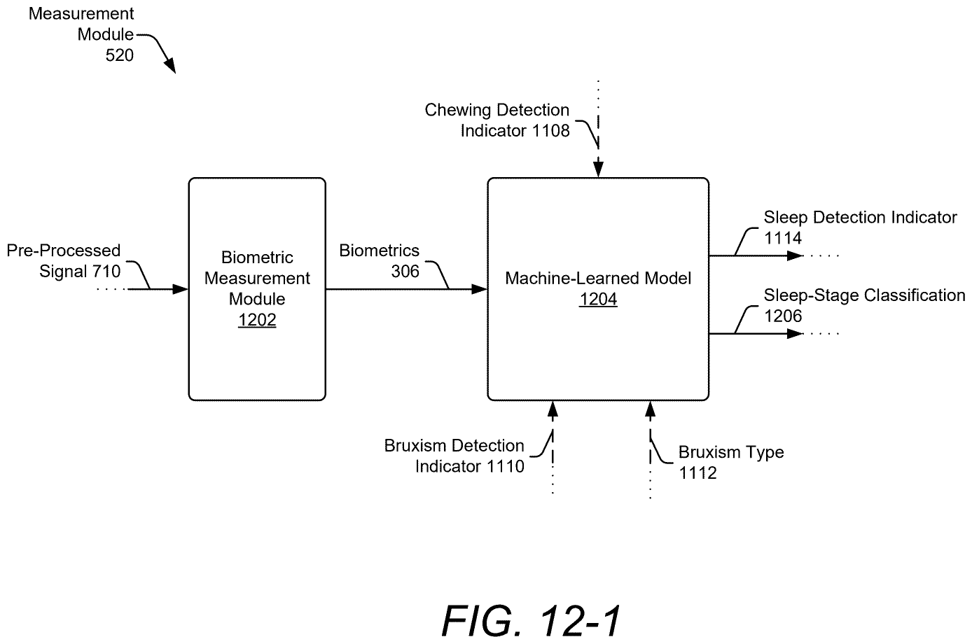

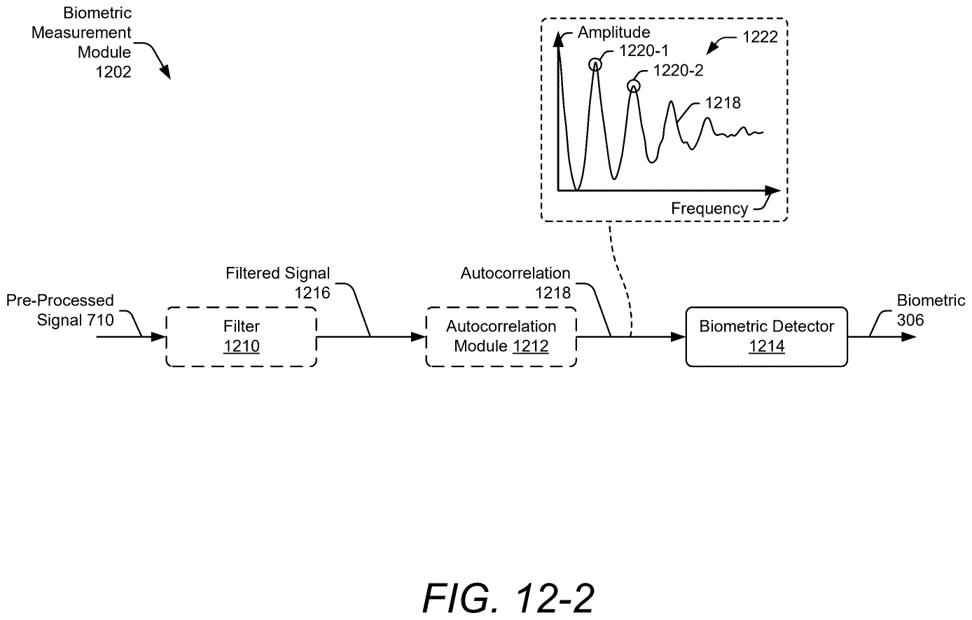

The hearable 102 can also perform interdependent human behavior detection and/or classification by using at least one of the above detected and/or classified human behaviors (e.g., chewing, bruxism, or sleep) to assist with detecting and/or classifying another one of the above human behaviors. For example, chewing detection 112, bruxism detection 114, and/or bruxism classification 116 can be used as an input for performing sleep detection 118 and/or sleep classification 120, as further described with respect to FIG. 12-1. As another example, sleep detection 118 can be used as an input for performing bruxism detection 114 and/or for performing chewing detection 112, as further described with respect to FIG. 11. Generally speaking, the detection of a behavior can refer to detecting an occurrence of the behavior or detecting an absence of the behavior.

Based on the information provided by detecting and/or classifying human behavior, a device (e.g., the hearable 102 and/or the computing device 104) can take additional actions to assist the user 106 in improving their health. An example action can include communicating this information to the user 106 or providing this information to an application or another entity specified by the user 106. Other example actions can include sounding an alarm, recommending a lifestyle change, suggesting music or background noise for reducing stress or improving sleep, and so forth. In some cases, the user 106 may choose to seek medical advice or make changes to their lifestyle based on the information provided by human behavior detection and/or classification.

To effectively use audioplethysmography 110 for detecting and/or classifying human behavior, the user 106 positions the hearable 102 in a manner that creates at least a partial seal 122 around or in the ear 108. Some parts of the ear 108 are shown in FIG. 1-1, including the ear canal 124 and an ear drum 126 (or tympanic membrane). Due to the seal 122, the hearable 102, the ear canal 124, and the ear drum 126 couple together to form an acoustic circuit. Audioplethysmography 110 involves, at least in part, measuring properties associated with this acoustic circuit. The properties of the acoustic circuit can change due to a variety of different situations or actions.

For example, consider FIG. 1-2 in which a change occurs in a physical structure of the ear 108. Example changes to the physical structure include a change in a geometric shape of the ear canal 124 and/or a change in a volume of the ear canal 124. This change can be caused, at least in part, by subtle blood vessel deformations in the ear canal 124 caused by the user 106's heart pumping. Other changes can also be caused by the user 106's breathing, movement of the user 106's jaw, and/or other movements made by the user 106.

At 128, for instance, the tissue around the ear canal 124 and the ear drum 126 itself are slightly “squeezed” due to blood vessel deformation. This squeeze causes a volume of the ear canal 124 to be slightly reduced at 128. At 130, however, the squeezing subsides and the volume of the ear canal 124 is slightly increased relative to 128. The physical changes within the ear 108 can modulate an amplitude and/or phase of an acoustic signal that propagates through the ear canal 124, as further described below.

During audioplethysmography 110, an acoustic signal propagates through at least a portion of the ear canal 124. The hearable 102 can receive an acoustic signal that represents a superposition of multiple acoustic signals that propagate along different paths within the ear canal 124. Each path is associated with a delay (i) and an amplitude (a). The delay and amplitude can vary over time due to the subtle changes that occur in the volume of the ear canal 124. The received acoustic signal can be represented by Equation 1:

$$S ( t ) = n + {\sum }_{i = 1}^{N - 1} {a}_{i} ( t ) cos ( {φ}_{i n i} + {Ω}_{f c} ( t + {τ}_{i} ( t ) ) ) \qquad Equation 1$$

where S(t) represents the received acoustic signal, n represents noise, φᵢₙᵢ represents a relative phase between the received acoustic signal and the transmitted acoustic signal, Ω_{fc} represents a frequency of the transmitted acoustic signal, and t represents a time vector. Biometrics and/or jaw movements of the user 106, for instance, can modulate the amplitude and/or phase of the receive acoustic signal, as further shown in Equation 2:

$$S ( t ) = n + ( 1 + {h}_{a m p} ( t ) ) cos ( {h}_{p h a s e} ( t ) + {φ}_{i n i} + + {Ω}_{f c} ( t ) ) \qquad Equation 2$$

where hₐₘₚ(t) represents an amplitude modulator and hₚₕₐₛₑ(t) represents a phase modulator. The interactions between the hearable 102 and the ear 108 as well as the physiological activities of the user 106 modulate the amplitude and phase of the received acoustic signal. The techniques for audioplethysmography 110 can be performed while the hearable 102 is playing audible content to the user 106. With active acoustic sensing, the hearable 102 can detect and/or classify various human behaviors, as further described with respect to FIGS. 2-1 to 3-2.

FIG. 2-1 illustrates an example environment 200-1 in which chewing detection 112 can be performed using active acoustic sensing. In the environment 200-1, the user 106 eats breakfast while wearing at least one hearable 102. The hearable 102 uses audioplethysmography 110 to perform chewing detection 112. The chewing detection 112 determines that the user 106 is chewing (or eating) in the environment 200-1.

In general, chewing detection 112 can also be used to capture a time of day in which the user 106 starts and stops eating. With this information, the hearable 102 and/or the computing device 104 can keep track of the user 106's eating habit. This can include determining when and/or how often the user 106 eats a meal or a snack. In some implementations, chewing detection 112 can estimate the user 106's calorie intake based on the duration of the chewing activity.

Using chewing detection 112 to monitor the user 106's eating habit can be particularly helpful for automatically tracking intermittent fasting and/or snacking. The user 106 can later review this information to determine how well they adhered to an intermittent fasting plan or how often they are snacking. In some implementations, the user 106 can enable a setting on the hearable 102 and/or the computing device 104 to cause a sound or music to be played if the user 106 forgot to eat within a certain time window. Additionally or alternatively, the user 106 can enable an alarm to discourage snacking. If chewing detection 112 determines that the user 106 is snacking between meals, for instance, the hearable 102 and/or the computing device 104 can sound the alarm to make the user 106 aware of the snacking. This may be helpful to enable the user 106 to reduce and/or break a snacking habit.

Chewing detection 112 can also be used to train children to properly chew their food before swallowing. For example, the hearable 102 and/or the computing device 104 can play audio content for the child as the child chews their food and play a sound once the child chews a target number of times before swallowing.

Generally speaking, chewing can involve a different type of jaw motion compared to bruxism. In one aspect, chewing involves the rhythmical movement of the jaw and/or tongue. The user's jaw can move up and down as well as from side to side to assist with grinding food. Audioplethysmography 110 can be used to detect the subtle differences between chewing and bruxism. Bruxism detection 114 and/or classification 116 are further described with respect to FIGS. 2-2 and 2-3.

FIG. 2-2 illustrates example environments 200-2 and 200-3 in which bruxism detection 114 and/or bruxism classification 116 can be performed using active acoustic sensing. In the environment 200-2, the user 106 works at a desk while wearing at least one hearable 102. In the environment 200-3, the user 106 sleeps while wearing at least one hearable 102. In both environments 200-2 and 200-3, the hearable 102 uses audioplethysmography 110 to perform bruxism detection 114 and/or bruxism classification 116.

With bruxism detection 114, the hearable 102 can determine the frequency and duration of bruxism. As the user 106 may be more likely to wear a hearable 102 throughout the day compared to another bruxism-detection sensor that adheres to the user 106's face, the hearable 102 can be used to monitor for bruxism throughout the day (e.g., while the user is working or while the user is in public). As such, the hearable 102 can provide a more complete history of the occurrences of bruxism compared to other sensors that are only wom at night or while the user 106 is home.

Using bruxism detection 114 and/or bruxism classification 116 to automatically monitor bruxism can be particularly helpful for evaluating the user 106's stress levels, determining the quality of the user 106's sleep, and/or monitoring the progression of bruxism related issues, such as temporomandibular disorder. The user 106 can later review this information to determine whether steps they have taken to reduce bruxism are helping or not.

In some implementations, the user 106 can enable an alarm to prevent bruxism. If bruxism detection 114 determines that bruxism is occurring, for instance, the hearable 102 and/or the computing device 104 can sound the alarm to make the user 106 aware of the bruxism and stop the behavior. This alarm may allow the user 106 to train themselves to reduce or stop bruxism. In this sense, bruxism detection 114 enables the user 106 to become conscious of the occurrence of unconscious bruxism, thereby enabling them to break the behavior. Various types of bruxism that can be identified using bruxism classification 116 are further described with respect to FIG. 2-3.

FIG. 2-3 illustrates various types of bruxism 202, which can be detected and/or classified using active acoustic sensing. Example types of bruxism 202 include clenching 204 (or pulsing), tapping 206, and grinding 208. Clenching 204 involves the user 106 clenching their jaw or biting down such that force is applied between the upper and lower jaw. Often times clenching 204 involves the user 106's jaw remaining in this closed state for a longer period of time compared to chewing. Sometimes clenching 204 can indicate an emotional state of the user 106, such as a state of anger, determination, and/or stress. In general, clenching 204 can lead to pain and/or fatigue in the jaw region.

Tapping 206 involves the user 106 tapping their upper and lower jaw together. The force applied during tapping 206 can be less than the force applied during clenching 204. Additionally or alternatively, a duration in which the user 106 bites down during tapping 206 can be shorter compared to clenching 204. In general, tapping 206 can lead to worn down or broken teeth.

Grinding 208 involves the user 106 moving their jaw in a manner that causes the user's teeth to grate or scrape across each other. Different types of grinding 208 can be associated with different directions in which the jaw moves. Side-to-side grinding 208-1, for instance, can involve the user 106's teeth scraping across each other in a left-to-right (or right-to-left) manner. Front-to-back grinding 208-2 can involve the user 106's teeth scraping across each other in a front-to-back (or back-to-front) manner. In general, grinding 208 can lead to worn down or broken teeth.

By detecting the various types of bruxism 202, bruxism classification 116 can improve the performance of bruxism detection 114 by identifying false positives associated with other human behaviors, such as chewing. Additionally or alternatively, bruxism classification 116 can be used to evaluate the progression of bruxism-related issues, such as temporomandibular disorder, and/or identify solutions to help prevent bruxism 202.

FIG. 3-1 illustrates an example environment 300 in which sleep detection 118 and/or sleep classification 120 can be performed using active acoustic sensing. In the environment 300, the user 106 goes to sleep while wearing at least one hearable 102. The hearable 102 uses audioplethysmography 110 to perform sleep detection 118 and/or sleep classification 120.

Sleep detection 118 can determine whether the user 106 is awake or sleeping. With this information, the sleep detection 118 can monitor how often the user 106 sleeps, when the user 106 falls asleep, and/or how long the user 106 sleeps. Sleep classification 120 can further identify the various stages of sleep. With this information, sleep classification 120 can determine how long the user 106 spends in each sleep stage and estimate the quality of the sleep.

A typical sleep cycle 302 is illustrated at the bottom of FIG. 3-1. The sleep cycle 302 includes four stages 304-1, 304-2, 304-3, and 304-4. A first stage 304-1 (or N1) represents a stage in which the user 106 begins to fall asleep. During the second stage 304-2 (or N2), the user 106 is lightly sleeping. In the second stage 304-2, the user 106's body temperature may begin to drop, their muscles may relax, and their respiration rate and/or heart rate may slow. During the third stage 304-3 (or N3), the user 106 is in a deep sleep. The user 106's body further relaxes and their heart rate and/or respiration rate can further slow relative to the second stage 304-2. A fourth stage 304-4 (or N4) includes rapid eye movement (REM) sleep. During the fourth stage 304-4, the user 106's body may be relatively stationary except for the user 106's eyes and/or breathing muscles.

To perform sleep detection 118 and/or sleep classification 120, active acoustic sensing monitors and/or measures two or more biometrics of the user 106. Example biometrics are further described with respect to FIG. 3-2.

FIG. 3-2 illustrates example biometrics 306 that can be monitored or measured using active acoustic sensing. Two or more of these biometrics 306 can be used for sleep detection 118 and/or sleep classification 120. Example biometrics 306 include the user 106's heart rate 308, respiration rate 310, blood pressure 312, occurrence (or absence) of bruxism 202, the occurrence (or absence) of coughing 314, the occurrence (or absence) of muscle movement 316, and the occurrence (or absence) of chewing (now shown). Although labeled as biometrics 306 in FIG. 3-2, bruxism 202, coughing 314, and/or chewing also represent other human behaviors that can be detected using active acoustic sensing.

The heart rate 308 can include the user 106's current heart rate 308, the user 106's resting heart rate, and/or the user 106's heart-rate variability. The respiration rate 310 can include the user 106's current respiration rate 310, the user 106's resting respiration rate, and/or the user 106's respiration-rate variability. Example muscle movements 316 that can be detected using active acoustic sensing include head movement 318 and/or eye movement 320. Large and/or frequent head movements 318 can indicate poor sleep quality. Bruxism 202 and/or coughing 314 can also be an indication of poor sleep quality. Eye movement 320 can indicate whether the user 106 is awake or asleep. The eye movement 320 can also be used to classify different stages 304 of sleep. The fourth stage 304-4, for instance, can include a significant amount of rapid eye movement 320 compared to other sleep stages 304. The detection of chewing can indicate that the user 106 is awake and not asleep. The biometrics 306 can also be used to further determine a quality of the user 106's sleep.

In comparison to other types of sensors, active acoustic sensing using the hearable 102 can be a more comfortable, convenient, and cost effective means of monitoring sleep behavior. Furthermore, active acoustic sensing can provide better quality sleep data compared to other sensors that are positioned further away from the user 106's head or positioned at a remote location. Consider another monitoring device that measures movement of the user 106's chest to determine the respiration rate. This method of measuring the respiration rate can be less accurate compared to the techniques associated with audioplethysmography 110. There may also be situations in which the user 106's chest is obscured by a pillow or another object.

Some sleep-monitoring devices may be unable to measure at least some of the biometrics 306 and/or detect other human behaviors that audioplethysmography 110 is capable of measuring and/or detecting. With limited data, sleep quality analysis provided by these other sensors may be less accurate compared to the sleep quality analysis provided using techniques associated with audioplethysmography 110.

Other challenges with monitoring sleep can include distinguishing between multiple people that are sleeping in a same room or a same bed. While some devices may use a microphone to detect noises associated with sleep, such as snoring, it can be challenging of the microphone to determine whether the noise is coming from the intended user 106 or from another person who is also in the room. In contrast, the techniques for audioplethysmography 110 can readily monitor a particular user 106's sleeping habit even if there are multiple people sleeping in the same room.

Based on the information provided by chewing detection 112, bruxism detection 114, bruxism classification 116, sleep detection 118, and/or sleep classification 120, the user 106 can choose to seek medical advice or make changes to their lifestyle. This information can also be used to control an operation of the computing device 104, which is further described with respect to FIG. 4.

FIG. 4 illustrates an example implementation of the computing device 104. The computing device 104 is illustrated with various non-limiting example devices including a desktop computer 104-1, a tablet 104-2, a laptop 104-3, a television 104-4, a computing watch 104-5, computing glasses 104-6, a gaming system 104-7, a microwave 104-8, and a vehicle 104-9. Other devices may also be used, such as an augmented and/or virtual reality headset, a home service device, a smart speaker, a smart thermostat, a baby monitor, a Wi-Fi™ router, a drone, a trackpad, a drawing pad, a netbook, an e-reader, a home automation and control system, a wall display, and another home appliance. Note that the computing device 104 can be wearable, non-wearable but mobile, or relatively immobile (e.g., desktops and appliances).

The computing device 104 includes one or more computer processors 402 and at least one computer-readable medium 404, which includes memory media and storage media. Applications and/or an operating system (not shown) embodied as computer-readable instructions on the computer-readable medium 404 can be executed by the computer processor 402 to provide some of the functionalities described herein. The computer-readable medium 404 can optionally include an application 406. The application 406 can use information provided by the hearable 102 to perform an action based on the human behavior detection and/or classification. Example actions can include displaying data, outputting data, compiling data, analyzing data, sounding an alarm, providing a recommendation for improving the user 106's health based on the data, and so forth.

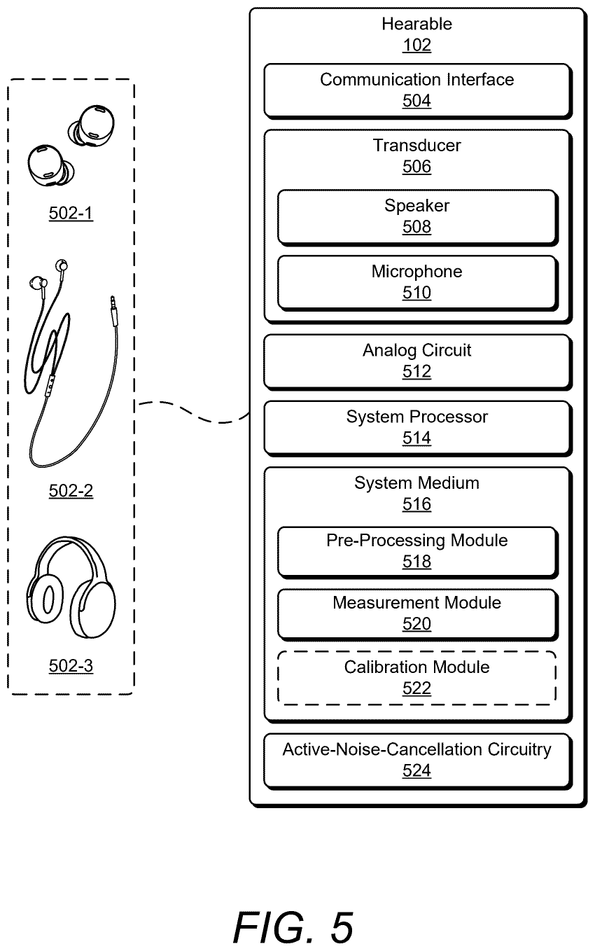

The computing device 104 can also include a network interface 408 for communicating data over wired, wireless, or optical networks. For example, the network interface 408 may communicate data over a local-area-network (LAN), a wireless local-area-network (WLAN), a personal-area-network (PAN), a wire-area-network (WAN), an intranet, the Internet, a peer-to-peer network, point-to-point network, a mesh network, Bluetooth®, and the like. The computing device 104 may also include the display 410. Although not explicitly shown, the hearable 102 can be integrated within the computing device 104, or can connect physically or wirelessly to the computing device 104. The hearable 102 is further described with respect to FIG. 5.

FIG. 5 illustrates an example hearable 102. The hearable 102 is illustrated with various non-limiting example devices, including wireless earbuds 502-1, wired earbuds 502-2, and headphones 502-3. The hearable 102 can also represent a hearing aid (not shown). The earbuds 502-1 and 502-2 are a type of in-ear device that fits into the ear canal 124. Each earbud 502-1 or 502-2 can represent a hearable 102. Headphones 702-3 can rest on top of or over the ears 108. The headphones 702-3 can represent closed-back headphones, open-back headphones, on-ear headphones, or over-ear headphones. Each headphone 702-2 includes two hearables 102, which are physically packaged together. In general, there is one hearable 102 for each ear 108. The headphones 402-3 may be designed in some manner or may utilize techniques, such as beamforming, to assist with directing signals used for audioplethysmography 110 into the ear canal 124.

The hearable 102 includes a communication interface 504 to communicate with the computing device 104, though this need not be used when the hearable 102 is integrated within the computing device 104. The communication interface 504 can be a wired interface or a wireless interface, in which audio content is passed from the computing device 104 to the hearable 102. The hearable 102 can also use the communication interface 504 to pass data associated with human behavior detection and/or classification to the computing device 104. In general, the data provided by the communication interface 504 is in a format usable by the application 406 or the computing device 104.

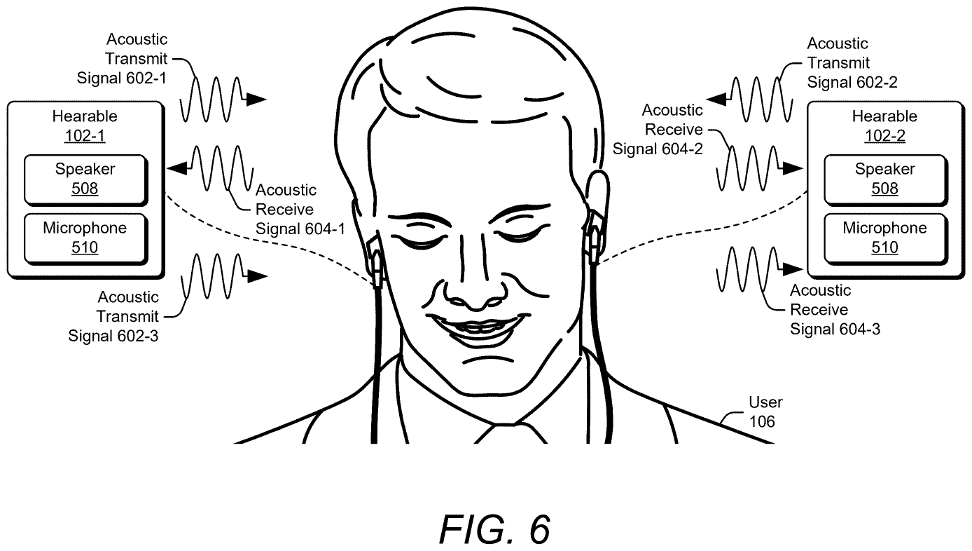

The communication interface 504 also enables the hearable 102 to communicate with another hearable 102. During bistatic sensing, for instance, the hearable 102 can use the communication interface 504 to coordinate with the other hearable 102 to support two-ear audioplethysmography 110, as further described with respect to FIG. 6. In particular, the transmitting hearable 102 can communicate timing and waveform information to the receiving hearable 102 to enable the receiving hearable 102 to appropriately demodulate a received acoustic signal.

The hearable 102 includes at least one transducer 506 that can convert electrical signals into sound waves. The transducer 506 can also detect and convert sound waves into electrical signals. These sound waves may include ultrasonic frequencies and/or audible frequencies, either of which may be used for audioplethysmography 110. In particular, a frequency spectrum (e.g., range of frequencies) that the transducer 506 uses to generate an acoustic signal can include frequencies from a low-end of the audible range to a high-end of the ultrasonic range, e.g., between 20 hertz (Hz) to 2 megahertz (MHz). Other example frequency spectrums for audioplethysmography 110 can encompass frequencies between 20 Hz and 20 kilohertz (kHz), between 20 kHz and 2 MHz, between 20 and 96 kHz, between 20 and 60 kHz, or between 30 and 40 kHz.

In an example implementation, the transducer 506 has a monostatic topology. With this topology, the transducer 506 can convert the electrical signals into sound waves and convert sound waves into electrical signals (e.g., can transmit or receive acoustic signals). Example monostatic transducers may include piezoelectric transducers, capacitive transducers, and micro-machined ultrasonic transducers (MUTs) that use microelectromechanical systems (MEMS) technology.

Alternatively, the transducer 506 can be implemented with a bistatic topology, which includes multiple transducers that are physically separate. In this case, a first transducer converts the electrical signal into sound waves (e.g., transmits acoustic signals), and a second transducer converts sound waves into an electrical signal (e.g., receives the acoustic signals). An example bistatic topology can be implemented using at least one speaker 508 and at least one microphone 510. The speaker 508 and the microphone 510 can be dedicated for audioplethysmography 110 or can be used for both audioplethysmography 110 and other functions of the computing device 104 (e.g., presenting audible content to the user 106, capturing the user 106's voice for a phone call, or for voice control).

In general, the speaker 508 and the microphone 510 are directed towards the ear canal 124 (e.g., oriented towards the ear canal 124). Accordingly, the speaker 508 can direct acoustic signals towards the ear canal 124, and the microphone 510 can receive acoustic signals from the direction associated with the ear canal 124. In some cases, the hearable 102 includes another microphone 510 that is directed away from the ear canal 124 towards an external environment (e.g., oriented away from the ear canal 124). This other microphone can be used to receive over-the-air signals for active-noise-cancellation or a transparency mode.

The hearable 102 includes at least one analog circuit 512, which includes circuitry and logic for conditioning electrical signals in an analog domain. The analog circuit 512 can include analog-to-digital converters, digital-to-analog converters, amplifiers, filters, mixers, and switches for generating and modifying electrical signals. In some implementations, the analog circuit 512 includes other hardware circuitry associated with the speaker 508 or microphone 510.

The hearable 102 also includes at least one system processor 514 and at least one system medium 516 (e.g., one or more computer-readable storage media). In the depicted configuration, the system medium 516 includes a pre-processing module 518 and a measurement module 520. The system medium 516 also optionally includes a calibration module 522. The pre-processing module 518, the measurement module 520, and the calibration module 522 can be implemented using hardware, software, firmware, or a combination thereof. In this example, the system processor 514 implements the pre-processing module 518, the measurement module 520, and the calibration module 522. In an alternative example, the computer processor 402 of the computing device 104 can implement at least a portion of the pre-processing module 518, the measurement module 520, and/or the calibration module 522. In this case, the hearable 102 can communicate digital samples of the acoustic signals to the computing device 104 using the communication interface 504.

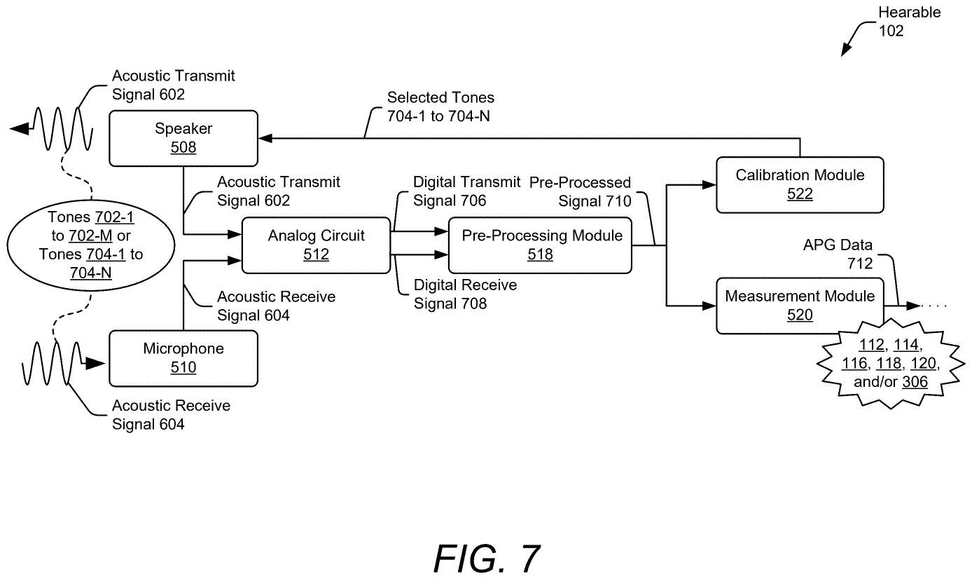

Operations of the pre-processing module 518, the measurement module 520, and the calibration module 522 are further described with respect to FIGS. 7 to 10. Aspects of detecting and/or classifying human behavior can be performed, at least partially, by the measurement module 520, as further described with respect to FIGS. 11 and 12. In other words, the measurement module 520 can perform aspects of chewing detection 112, bruxism detection 114, bruxism classification 116, sleep detection 118, and/or sleep classification 120. These features can be performed in parallel or in series. In some cases, the detection and/or classification of a first behavior is used to enhance (e.g., improve the accuracy and/or confidence level of) the detection and/or classification of a second behavior.

Some hearables 102 include the active-noise-cancellation circuitry 524, which enables the hearables 102 to reduce background or environmental noise. In this case, the microphone 510 used for audioplethysmography 110 can be implemented using a feedback microphone of the active-noise-cancellation circuitry 524. During active noise cancellation, the feedback microphone provides feedback information regarding the performance of the active noise cancellation. During audioplethysmography 110, the feedback microphone receives an acoustic signal, which is provided to the pre-processing module 518. In some situations, active noise cancellation and audioplethysmography 110 are performed simultaneously using the feedback microphone. In this case, the acoustic signal received by the feedback microphone can be provided to the pre-processing module 518 and the feedback signal for active noise cancellation can be provided to the active-noise-cancellation circuitry 524.

The hearable 102 can also include other auxiliary sensors, such as a motion sensor. Example motion sensors include an inertial measurement unit (IMU), an accelerometer, an inclinometer, a gyroscope, a magnetometer, a Global Navigation Satellite System (GNSS), or some combination thereof. In general, the motion sensor can detect and/or measure one or more characteristics of motion. Some motion sensors, for instance, can measure linear acceleration and/or a rotational velocity (or angular velocity), detect changes in orientation, detect changes in inclination, or some combination thereof. The linear accelerations and the rotational velocities can be associated with one, two, or three orthogonal axes. The motion sensor can generate motion-sensing data for audioplethysmography 110. The motion-sensing data can include time-series data associated with the measured linear accelerations and/or rotational velocities. Other types of motion-sensing data can include indications of changes in orientation and/or inclination, coordinates measured by the Global Navigation Satellite System, and so forth. The motion-sensing data can include one or more of the characteristics of motion described above.

The motion-sensing data can be utilized by audioplethysmography to perform motion-artifact filtering and/or activity detection. With motion-artifact filtering, noise caused by motion of the user 106 can be attenuated to improve sensitivity and accuracy for audioplethysmography 110. For example, audioplethysmography 110 can utilize motion-artifact filtering to accurately measure the user 106's heart rate while the user 106 is jogging. Motion-artifact filtering can also be used to reduce a false-alarm rate or false detections associated with other use cases of audioplethysmography 110, such as chewing detection 112. Other hearables that do not utilize motion-artifact filtering may be unable to accurately collect data using audioplethysmography 110 while the user 106 is moving, which can significantly limit the usefulness of audioplethysmography 110 and present an inconvenience for the user 106.

Activity detection uses audioplethysmography 110 and the motion-sensing data to determine that the user 106 is moving. Information about when and how often the user 106 moves can provide additional data for a variety of different use cases. Sleep quality analysis, for instance, can utilize this information to estimate how well the user 106 slept. Different types of audioplethysmography 110 are further described with respect to FIG. 6.

### Active Acoustic Sensing

FIG. 6 illustrates example operations of two hearables 102-1 and 102-2. In a first example operation, the hearables 102-1 and 102-2 perform single-ear audioplethysmography 110. This means that the hearables 102-1 and 102-2 independently perform audioplethysmography 110 on different ears 108 of the user 106. In this case, the first hearable 102-1 is proximate to the user 106's right ear 108, and the second hearable 102-2 is proximate to the user 106's left ear 108. Each hearable 102-1 and 102-2 includes a speaker 508 and a microphone 510. The hearables 102-1 and 102-2 can operate in a monostatic manner during the same time period or during different time periods. In other words, each hearable 102-1 and 102-2 can independently transmit and receive ultrasound signals.

For example, the first hearable 102-1 uses the speaker 508 to transmit a first acoustic transmit 602-1, which propagates within at least a portion of the user 106's right ear canal 124. The first hearable 102-1 uses the microphone 510 to receive a first acoustic receive signal 604-1. The first acoustic receive signal 604-1 represents a version of the first acoustic transmit signal 602-1 that is modified, at least in part, by the acoustic circuit associated with the right ear canal 124. This modification can change an amplitude, phase, and/or frequency of the first acoustic receive signal 604-1 relative to the first acoustic transmit signal 602-1.

Similarly, the second hearable 102-2 uses the speaker 508 to transmit a second acoustic transmit signal 602-2, which propagates within at least a portion of the user 106's left ear canal 124. The second hearable 102-2 uses the microphone 510 to receive a second acoustic receive signal 604-2. The second acoustic receive signal 604-2 represents a version of the second acoustic transmit signal 602-2 that is modified by the acoustic circuit associated with the left ear canal 124. This modification can change an amplitude, phase, and/or frequency of the second acoustic receive signal 604-2 relative to the second acoustic transmit signal 602-2.

The techniques of single-ear audioplethysmography 110 can be particularly beneficial as it enables the computing device 104 to compile information from both hearables 102-1 and 102-2, which can further improve measurement confidence. For some aspects of audioplethysmography 110, it can be beneficial to analyze the acoustic channel between two ears 108, as further described below.

In a second example operation, the two hearables 102-1 and 102-2 perform two-ear audioplethysmography 110. This means that the hearables 102-1 and 102-2 jointly perform audioplethysmography 110 across two ears 108 of the user 106. In this case, at least one of the hearables 102 (e.g., the first hearable 102-1) includes the speaker 508, and at least one of the other hearables 102 (e.g., the second hearable 102-2) includes the microphone 510. The hearables 102-1 and 102-2 operate together in a bistatic manner during the same time period.

During operation, the first hearable 102-1 transmits a third acoustic transmit signal 602-3 using the speaker 508. The third acoustic transmit signal 602-3 propagates through the user 106's right ear canal 124. The third acoustic transmit signal 602-3 also propagates through an acoustic channel that exists between the right and left ears 108. In the left ear 108, the third acoustic transmit signal 602-3 propagates through the user 106's left ear canal 124 and is represented as a third acoustic receive signal 604-3. The second hearable 102-2 receives the third acoustic receive signal 604-3 using the microphone 510. The third acoustic receive signal 604-3 represents a version of the third acoustic transmit signal 602-3 that is modified by the acoustic circuit associated with the right ear canal 124, modified by the acoustic channel associated with the user 106's face, and modified by the acoustic circuit associated with the left ear canal 124. This modification can change an amplitude, phase, and/or frequency of the third acoustic receive signal 604-3 relative to the third acoustic transmit signal 602-3. In some cases, the hearable 102-2 measures the time-of-flight (ToF) associated with the propagation from the first hearable 102-1 to the second hearable 102-2. Sometimes a combination of single-ear and two-ear audioplethysmography 110 are applied to further improve measurement confidence.

The acoustic transmit signals 602 of FIG. 6 can represent a variety of different types of signals as described above with respect to FIG. 5. In example implementations, the acoustic transmit signal 602 can be the ultrasound signal. Also, the acoustic transmit signal 602 can be a continuous-wave signal (e.g., a sinusoidal signal) or a pulsed signal. Some acoustic transmit signals 602 can have a particular tone (or frequency). Other acoustic transmit signals 602 can have multiple tones (or multiple frequencies). A variety of modulations can be applied to generate the acoustic transmit signal 602. Example modulations include linear frequency modulations, triangular frequency modulations, stepped frequency modulations, phase modulations, or amplitude modulations. The acoustic transmit signal 602 can be transmitted as part of a calibration procedure or a measurement procedure, as further described as part of FIG. 7.

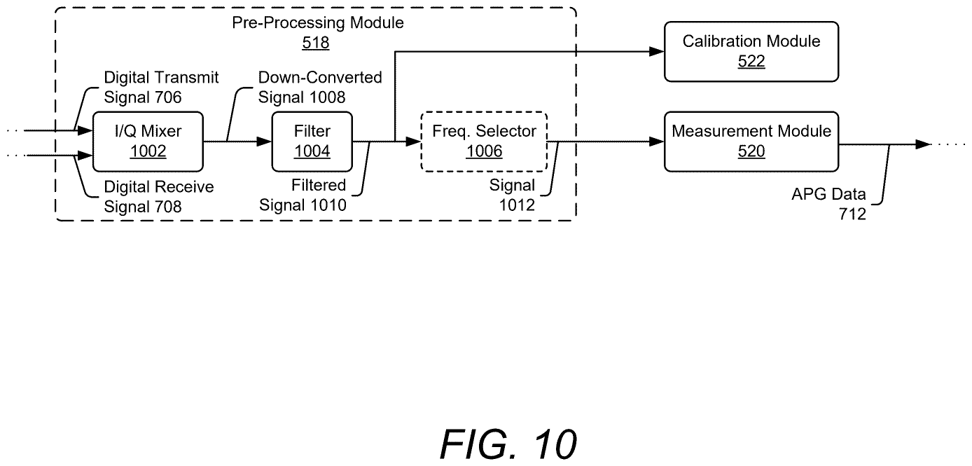

FIG. 7 illustrates an example implementation of the hearable 102 for detecting and/or classifying human behavior using active acoustic sensing. In the depicted configuration, the hearable 102 includes the speaker 508, the microphone 510, the analog circuit 512, the pre-processing module 518, the measurement module 520, and the calibration module 522. Other implementations of the hearable 102, however, are also possible in which the hearable 102 does not include the calibration module 522 to reduce processing power requirements. In this case, the pre-processing module 518 can perform aspects of frequency selection as further described with respect to FIG. 10 to improve the signal-to-noise ratio for audioplethysmography 110.

Outputs of the speaker 508 and the microphone 510 are coupled to inputs of the analog circuit 512. The pre-processing module 518 has inputs that are coupled to outputs of the analog circuit 512. The pre-processing module 518 also has outputs that are coupled to inputs of the measurement module 520 and the calibration module 522. The calibration module 522 has an output that is coupled to the speaker 508.

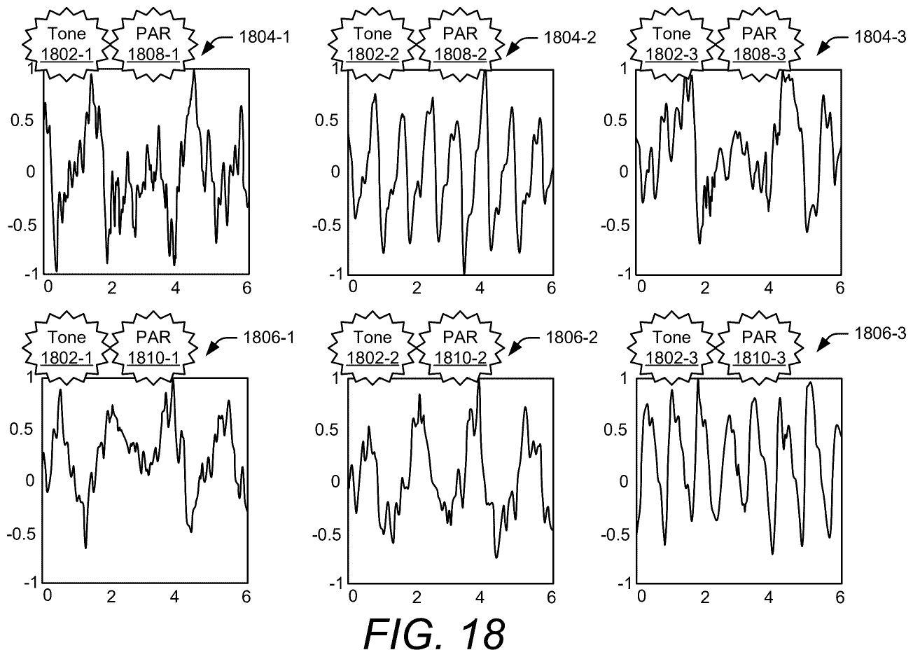

Consider an example operation of the hearable 102 in accordance with single-ear audioplethysmography 110. In the case that the hearable 102 includes the calibration module 522, the hearable 102 can perform a calibration process prior to performing a measurement process. The calibration process and the measurement process are further described with respect to FIG. 18.

During both the calibration process and the measurement process, the speaker 508 transmits the acoustic transmit signal 602 and the microphone 510 receives the acoustic receive signal 604. During the calibration process, the acoustic transmit signal 602 and the acoustic receive signal 604 can have tones 702-1 to 702-M, where M represents a positive integer. During the measurement process, the acoustic transmit signal 602 and the acoustic receive signal 604 can have selected tones 704-1 to 704-N, where N represents a positive integer that is less than or equal to M. The selected tones 704-1 to 704-N can represent a subset (sometimes a proper subset) of the tones 702-1 to 702-M.

The analog circuit 512 performs analog-to-digital conversion to generate a digital transmit signal 706 and a digital receive signal 708 based on the acoustic transmit signal 602 and the acoustic receive signal 604, respectively. In this sense, the digital transmit signal 706 represents a version of the acoustic transmit signal 602 and the digital receive signal 708 represents a version of the acoustic receive signal 604. The pre-processing module 518 performs frequency downconversion and demodulation to generate at least one pre-processed signal 710 based on the digital transmit signal 706 and the digital receive signal 708. The pre-processing module 518 can also apply filtering to generate the pre-processed signal 710.

As part of the calibration procedure, the calibration module 522 processes the pre-processed signal 710 to determine the selected tones 704-1 to 704-N. The selected tones 704-1 to 704-N can improve performance of audioplethysmography 110 during the measurement procedure. The calibration module 522 communicates the selected tones 704-1 to 704-N to the speaker 508 using a control signal. The speaker 508 accepts the control signal that identifies the selected tones 704-1 to 704-N and can transmit a subsequent acoustic transmit signal 602 for the measurement procedure using the selected tones 704-1 to 704-N.

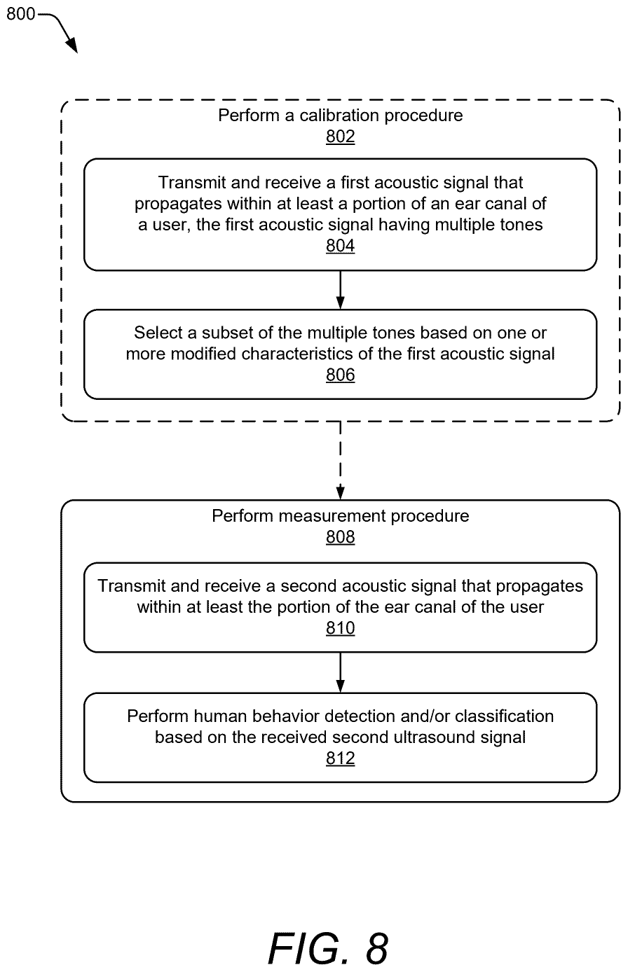

As part of the measurement procedure, the measurement module 520 can detect and/or classify various human behaviors using the pre-processed signal 710. In other words, the measurement module 520 can also perform aspects of chewing detection 112, bruxism detection 114, bruxism classification 116, sleep detection 118, sleep classification 120, and/or biometric monitoring to generate audioplethysmography data 712 (APG data 712). For chewing detection 112, the audioplethysmography data 712 can include an indication of whether or not chewing is detected. For bruxism detection 114, the audioplethysmography data 712 can include an indication of whether or not bruxism is detected. For bruxism classification 116, the audioplethysmography data 712, can include an indication of the type of bruxism that is detected. For sleep detection 118, the audioplethysmography data 712 can include an indication of whether or not the user 106 is determined to be asleep. For sleep classification 120, the audioplethysmography data 712 can include an indication of a sleep stage that is detected. For biometric monitoring, the audioplethysmography data 712 can include information about one or more measured biometrics 306. Additionally or alternatively, the audioplethysmography data 712 can include a control signal for controlling operation of the hearable 102 and/or the computing device 104. The calibration procedure and the measurement procedure are further described with respect to FIG. 8.

FIG. 8 illustrates an example flow diagram 800 for operating a hearable 102. In FIG. 8, the hearable 102 can optionally perform a calibration procedure at 802 using the calibration module 522. The calibration procedure can determine appropriate characteristics (e.g., waveform or signal characteristics) of acoustic transmit signals 602 to improve audioplethysmography 110 (e.g., to enhance the performance of human behavior detection and/or classification). The calibration procedure enables audioplethysmography 110 to take into account the wear of the hearable 102 (e.g., the position of the hearable 102 relative to the ear canal 124) and the physical structure of the ear canal 124 to determine a transmission frequency that can increase sensitivity. With the calibration procedure, the hearable 102 can dynamically adjust the transmission frequency (e.g., one or more carrier frequencies) each time the seal 122 is formed (e.g., based on the wear of the hearable 102) and based on the unique physical structure of the ear 108. Through this calibration procedure, the hearables 102 on different ears 108 may operate with one or more different ultrasound frequencies. Steps of the calibration procedure are further described below.

In some circumstances, the hearable 102 can perform on-head detection (or in-ear detection) by detecting the presence of the seal 122 and initiating the calibration procedure based on a determination that on-head detection is “true.” In other circumstances, the hearable 102 can initiate the calibration procedure based on a specified schedule or a timer, which can be controlled by the user 106 via the computing device 104.

At 804, the hearable 102 executes the calibration procedure by transmitting and receiving a first acoustic signal. The first acoustic signal propagates within at least a portion of the ear canal 124 of the user 106 and has multiple tones 902-1 to 902-M (or multiple carrier frequencies). The multiple tones 702-1 to 702-M are transmitted in parallel or in series over a given time interval. The first acoustic transmit signal 602 can have a particular bandwidth on the order of several kilohertz. For example, the acoustic transmit signal 602 can have a bandwidth of approximately 4, 5, 6, 8, 10, 16, or 20 kHz. In example implementations, the first acoustic transmit signal 602 is transmitted over multiple seconds, such as 2, 3, 4, 6, or more seconds. A duration of each tone 702 can be evenly divided over a total duration of the first acoustic transmit signal 602.

In an example implementation, the acoustic transmit signal 602 has seven tones 702 (e.g., M equals 7). In some cases, the tones 702 are evenly distributed across an interval. For example, the tones 702 can be in 1 kHz increments between 32 kHz and 38 kHz (e.g., at approximately 32, 33, 34, 35, 36, 37, and 38 kHz). The term “approximately” means that the tones 702 can be within 5% of a given value or less (e.g., within 3%, 2%, or 1% of the given value).

An amplitude of the acoustic transmit signal 602 can be approximately the same across the tones 702-1 to 702-M. In this manner, power is evenly distributed across each tone 702. The quantity of tones 702 (e.g., M) can be determined based on an output power of the speaker 508. Increasing the quantity of tones 702 can increase a likelihood that the hearable 102 can support human behavior detection and/or classification across various conditions including user wear and a physical structure of the user 106's ear canal 124. However, an amplitude of the acoustic transmit signal 602 can be limited across these tones 702 based on the output power of the speaker 508. Thus, the quantity of tones 702 can be optimized based on an amount of output power that is available for audioplethysmography 110.

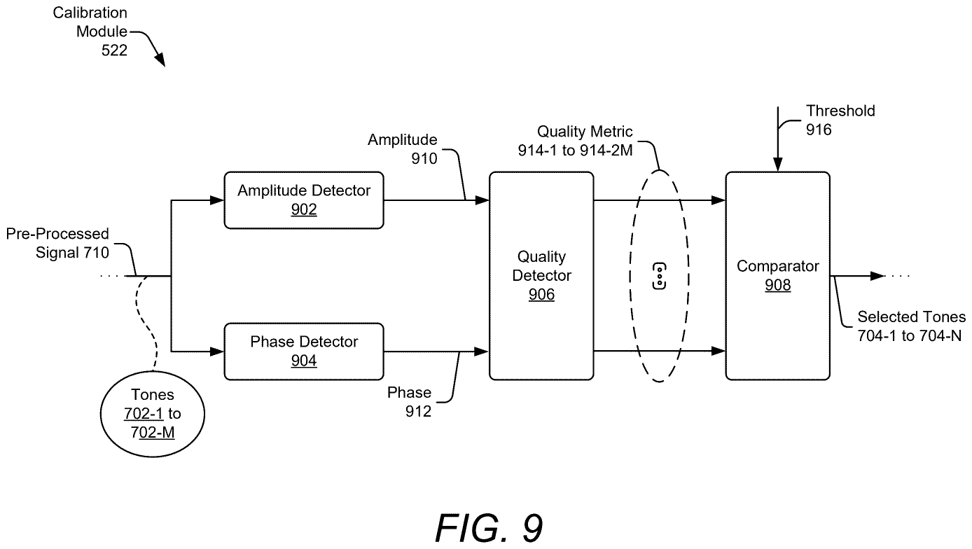

At 806, the calibration procedure selects one or more tones 704-1 to 704-N to be used for a measurement procedure based on one or more modified characteristics of the acoustic receive signal 604. The process for selecting the tones 704 is further described with respect to FIG. 9. In general, the calibration procedure determines that the selected tones 704 improve a signal-to-noise ratio for audioplethysmography 110 (or more specifically for human behavior detection and/or classification).

At 808, the hearable 102 performs a measurement procedure using the measurement module 520. In accordance with the measurement procedure, the hearable 102 transmits a second acoustic transmit signal 602 that propagates within at least the portion of the ear canal 124 of the user 106. If the calibration procedure was performed, the second acoustic transmit signal 602 can have the selected tones 704-1 to 704-M that were determined by the calibration procedure. The selected tones 704 can be transmitted in parallel or in series over a given time interval.

An amplitude of the second acoustic transmit signal 602 can be approximately the same across the selected tones 704-1 to 704-N. In this manner, power is evenly distributed across each selected tone 704. The amplitude of the second acoustic transmit signal 602 can be higher than the amplitude of the first acoustic transmit signal 602 because the available output power is distributed across fewer tones. Additionally or alternatively, a duration of each of the selected tones 704 of the second acoustic transmit signal 602 can be longer than the duration of the tones 702 of the first acoustic transmit signal 602. The higher amplitude and/or the longer duration can further improve the signal-to-noise ratio performance of the hearable 102 for audioplethysmography 110. By using a few selected tones 704 that were determined to improve signal-to-noise ratio performance, the measurement procedure can achieve a higher accuracy for human behavior detection and/or classification.

At 812, the hearable 102 performs audioplethysmography 110 (e.g., human behavior detection and/or classification) using the second acoustic signal (e.g., the second acoustic receive signal 604). One aspect of performing audioplethysmography 110 can include performing chewing detection 112, bruxism detection 114, bruxism classification 116, sleep detection 118, sleep classification 120, and/or biometric monitoring. The calibration module 522 is further described with respect to FIG. 10.

FIG. 9 illustrates an example scheme implemented by the calibration module 522. In the depicted configuration, the calibration module 522 implements a frequency selector, which selects one or more tones 704 for the measurement procedure. In the example implementation, the calibration module 522 includes at least one amplitude detector 902, at least one phase detector 904, at least one quality detector 906, and at least one comparator 908. The operations of these components are further described below.

During the calibration procedure, the calibration module 522 accepts the pre-processed signal 710 from the pre-processing module 518, as previously described with respect to FIG. 7. The pre-processed signal 710 can include amplitude and/or phase information associated with the multiple tones 702-1 to 702-M, which were used to transmit the first acoustic signal described at 802 in FIG. 8.

In this example, the calibration module 522 extracts an amplitude 910 of the pre-processed signal 710 using the amplitude detector 902 and extracts a phase 912 of the pre-processed signal 710 using the phase detector 904. Alternatively, if in-phase and quadrature components of the pre-processed signal 710 are received separately, the amplitude detector 902 and the phase detector 904 can respectively measure the amplitude 910 and phase 912 based on the in-phase and quadrature components.

The quality detector 906 measures quality metrics 914-1 to 914-2M for each of the tones 702-1 to 702-M and for each of the characteristics (e.g., amplitude 910 and phase 912). In general, the quality metrics 914 can represent a variety of different metrics, including peak-to-average ratios and/or signal-to-noise ratios. The peak-to-average ratio represents a peak intensity within a frequency range of interest divided by an average intensity within this frequency range. A higher quality metric 914 indicates a higher-quality signal, or more generally, better performance for audioplethysmography 110.

In one aspect, the comparator 908 can evaluate the quality metrics 914-1 to 914-2M with respect to a threshold 916. The threshold 916 can be set, for example, to a particular value. In other cases, the calibration module 522 can dynamically determine the threshold 916 and update it over time based on the observed quality metrics 914-1 of 914-2M. In an example implementation, the comparator 908 determines the selected tones 704-1 to 704-N for a subsequent measurement procedure based on the frequencies associated with the quality metrics 914-1 to 914-M that are greater than or equal to the threshold 916.

Additionally or alternatively, the comparator 908 can evaluate the quality metrics 914-1 to 914-2M with respect to each other. In an example implementation, the comparator 908 determines one of the selected tones 704 based on a frequency with the highest quality metric 914 across the amplitude 910. Also, the comparator 908 can determine one of the selected tones 704 based on a frequency with the highest quality metric 914 across the phase 912. In other implementations, the comparator 908 can determine a single selected tone 704 based on a frequency having the highest quality metric 914 associated with either the amplitude 910 or the phase 912.

In general, the calibration module 522 enables the selected tones 704-1 to 704-N to be dynamically adjusted prior to the measurement procedure based on a current environment, which can account for a wear orientation of the hearable 102 (e.g., a current insertion depth and/or rotation), a physical structure of the user 106's ear canal 124, and a response characteristic of the hearable 102 (e.g., speaker, microphone, and/or housing). In this manner, the calibration module 522 can improve the signal-to-noise ratio performance of the hearable 102 for the measurement procedure. The calibration module 522 can also determine which tones 704 generate acoustic receive signals 604 with desired characteristics for human behavior detection and/or classification.

In FIGS. 7 to 9, the calibration procedure and the measurement procedure are described as individual procedures that occur at different time intervals. In particular, the calibration procedure occurs before the measurement procedure. This enables the acoustic transmit signal 602 for the measurement procedure to be transmitted with fewer tones than the acoustic transmit signal 602 used for the calibration procedure, which can increase signal-to-noise ratio performance for audioplethysmography 110. In some implementations, however, the hearable 102 can have sufficient output power to perform the measurement procedure with the multiple tones 702-1 to 702-M using a single acoustic transmit signal 602. In this case, aspects of the calibration module can be integrated within the pre-processing module 518 as a frequency selector, which is further described with respect to FIG. 10. This frequency selector can effectively pass the selected tones 704-1 to 704-N for further processing. Aspects of the measurement procedure are further described with respect to FIG. 10.

FIG. 10 illustrates an example implementation of the pre-processing module 518 for performing aspects of active acoustic sensing. In the depicted configuration, the pre-processing module 518 includes at least one in-phase and quadrature mixer 1002 (IIQ mixer 1002) and at least one filter 1004. The in-phase and quadrature mixer 1002 performs frequency downconversion. In an example implementation, the in-phase and quadrature mixer 1002 includes at least two mixers, at least one phase shifter, and at least one combiner (e.g., a summation circuit). The filter 1004 attenuates intermodulation products that are generated by the in-phase and quadrature mixer 1002. In an example implementation, the filter 1004 is implemented using a low-pass filter.

The pre-processing module 518 can optionally include at least one frequency selector 1006. The frequency selector 1006 can identify and select one or more tones 704 (or carrier frequencies) that provide a high-quality signal for later processing. The frequency selector 1006 can further pass the selected tones to other processing modules (e.g., the measurement module 520) and filter (or attenuate) other tones that are not selected. The frequency selector 1006 can be implemented in a similar manner as the calibration module 522 of FIG. 9. For example, the frequency selector 1006, can include the amplitude detector 902, the phase detector 904, the quality detector 906, and the comparator 908.

During an operation, the in-phase and quadrature mixer 1002 uses the phase shifter and the two mixers to generate in-phase and quadrature components associated with the digital receive signal 708. In particular, the in-phase and quadrature mixer 1002 mixes the digital receive signal 708 with a first version of the digital transmit signal 706 that has a zero-degree phase shift to generate the in-phase component. Additionally, the in-phase and quadrature mixer 1002 mixes the digital receive signal 708 with a second version of the digital transmit signal 706 that has a 180-degree phase shift to generate the quadrature signal. This mixing operation downconverts the digital receive signal 708 from acoustic frequencies to baseband frequencies. Using the combiner, the in-phase and quadrature mixer 1002 combines the in-phase and quadrature components of the digital receive signal 708 to generate a down-converted signal 1008. Use of the in-phase and quadrature mixer 1002 can further improve the signal-to-noise ratio of the down-converted signal 1008 compared to other mixing techniques.

In this example, the down-converted signal 1008 represents a combination of the in-phase and quadrature components of the mixed-down digital receive signal 708. In alternative implementations, the in-phase and quadrature mixer 1002 doesn't include the combiner and passes the in-phase and quadrature components separately to the filter 1004. In this manner, the in-phase and quadrature components individually propagate through the filter 1004.

The filter 1004 generates a filtered signal 1010 based on the down-converted signal 1008. In particular, the filter 1004 filters the down-converted signal 1008 to attenuate spurious or undesired frequencies (e.g., intermodulation products), some of which can be associated with an operation of the in-phase and quadrature mixer 1002. In this example, the filtered signal 1010 represents a combination of the in-phase and quadrature components of the down-converted signal 1008. Alternatively, the filtered signal 1010 can represent separate or distinct in-phase and quadrature components, which are individually passed to the frequency selector 1006, the calibration module 522, or the measurement module 520.

During the measurement procedure, the pre-processing module 518 can optionally apply the frequency selector 1006. The frequency selector 1006 passes tones that meet a quality threshold level of performance for audioplethysmography 110. For example, the frequency selector 1006 passes tones 704 having an amplitude 910 and/or phase 912 with a quality metric 914 that is greater than or equal to a threshold 916. The resulting signal outputted by the frequency selector 1006 is represented by signal 1012. In some implementations, this signal 1012 is passed to the measurement module 520 as the pre-processed signal 710. In other implementations in which the frequency selector 1006 is not implemented, the filtered signal 1010 can be passed to the measurement module 520 and/or the calibration module 522 as the pre-processed signal 710.

In general, the measurement module 520 can generate the audioplethysmography data 712. In example implementations, the measurement module 520 can be implemented using a machine-learned model or another model that performs signal and/or data processing. Generally speaking, the measurement module 520 can analyze the changes in the amplitude 910 and/or phase 912 of the pre-processed signal 710 to detect and/or classify one or more human behaviors. Example components that can be used to implement the measurement module 520 are further described with respect to FIGS. 11 and 12-1.

Human Behavior Detection and/or Classification

FIG. 11 illustrates an example implementation of the measurement module 520 for performing chewing detection 112, bruxism detection 114, and/or bruxism classification 116. In the depicted configuration, the measurement module 520 is implemented using a machine-learned model 1100. In the example shown in FIG. 11, the machine-learned model 1100 includes a chewing detector 1102, a bruxism detector 1104, and a bruxism classifier 1106. The chewing detector 1102, the bruxism detector 1104, and the bruxism classifier 1106 can represent multiple machine-learned models or different stages of a single machine-learned model. Generally speaking, the chewing detector 1102 performs chewing detection 112, the bruxism detector 1104 performs bruxism detection 114, and the bruxism classifier 1106 performs bruxism classification 116. Other implementations of the machine-learned model 1100 can include a subset of the chewing detector 1102, the bruxism detector 1104, and/or the bruxism classifier 1106 depending on which human behaviors the hearable 102 is designed to detect and/or classify.

Each one of the chewing detector 1102, the bruxism detector 1104, and/or the bruxism classifier 1106 is implemented using one or more neural networks. A neural network includes a group of connected nodes (e.g., neurons or perceptrons), which are organized into one or more layers. As an example, the chewing detector 1102, the bruxism detector 1104, and/or the bruxism classifier 1106 can each include a deep neural network with an input layer, an output layer, and one or more hidden layers positioned between the input layer and the output layers. The nodes of the deep neural network can be partially-connected or fully-connected between the layers.

In some implementations, the neural network is a recurrent neural network (e.g., a long short-term memory (LSTM) neural network) with connections between nodes forming a cycle to retain information from a previous portion of an input data sequence for a subsequent portion of the input data sequence. In other cases, the neural network is a feed-forward neural network in which the connections between the nodes do not form a cycle. Additionally or alternatively, the chewing detector 1102, the bruxism detector 1104, and/or the bruxism classifier 1106 can include another type of neural network, such as a convolutional neural network. The chewing detector 1102, the bruxism detector 1104, and/or the bruxism classifier 1106 can include one or more types of classification models, such as a binary classification model, a multi-class classification model, multi-label classification, and so forth. Other implementations are also possible in which the chewing detector 1102, the bruxism detector 1104, and/or the bruxism classifier 1106 includes one or more types of regression models. In this case, the chewing detector 1102, the bruxism detector 1104, and/or the bruxism classifier 1106 can output a determined probability, likelihood, or confidence level associated with the occurrence of a corresponding human behavior and/or associated with a classification type of the corresponding human behavior.

Through supervised learning, the machine-learned model 1100 is trained to detect and/or classify one or more human behaviors associated with chewing and/or bruxism based on the pre-processed signal 710. In general, the supervised learning can use simulated (e.g., synthetic) data or measured (e.g., real) data for training purposes.

The chewing detector 1102 is trained to generate a chewing detection indicator 1108 based at least on the pre-processed signal 710. The chewing detection indicator 1108 can indicate whether or not chewing is detected within the pre-processed signal 710. In other words, the chewing detection indicator 1108 indicates the occurrence (or absence) of chewing.

The bruxism detector 1104 is trained to generate a bruxism detection indicator 1110 based at least on the pre-processed signal 710. The bruxism detection indicator 1110 can indicate whether or not bruxism 202 is detected within the pre-processed signal 710. In other words, the bruxism detection indicator 1110 indicates the occurrence (or absence) of bruxism 202.

The bruxism classifier 1106 is trained to generate a bruxism type 1112 based on the pre-processed signal 710 and the bruxism detection indicator 1110. The bruxism type 1112 can indicate a manner in which bruxism 202 manifests itself. For example, the bruxism type 1112 can indicate that the detected bruxism 202 involves clenching 204, tapping 206, and/or grinding 208 (e.g., side-to-side grinding 208-1 and/or front-to-back grinding 208-2). In some implementations, the bruxism classifier 1106 can also accept data from other sensors, such as a motion sensor.

The chewing detection indicator 1108, the bruxism detection indicator 1110, and/or the bruxism type 1112 can be provided to other processing entities, such as other processing modules or the application 406. In some aspects, these other processing entities can generate additional data based on the chewing detection indicator 1108, the bruxism detection indicator 1110, and/or the bruxism type 1112. Example data can include information for tracking the human behavior, such as times during which chewing and/or bruxism 202 occurred or a duration associated with the detected chewing and/or bruxism 202.

In other aspects, these other processing entities can utilize the information provided by chewing detection 112, bruxism detection 114, and/or bruxism classification 116 for detecting and/or classifying other human behaviors, such as sleep detection 118 and/or sleep classification 120 as further described with respect to FIG. 12-1. In a first example, a processing entity uses the chewing detection indicator 11108 to estimate the user 106's calorie intake or to determine if the user 106 followed a specified eating schedule. In a second example, the processing entity uses the bruxism detection indicator 1110 and/or the bruxism type 1112 to estimate the user 106's stress level. In a third example, the processing entity uses the bruxism detection indicator 1110 and/or the bruxism type 1112 for sleep detection 118 and/or sleep classification 120, as further described with respect to FIG. 12-1. This is an example of interdependent human behavior detection and/or classification.

Another example implementation of interdependent human behavior detection and/or classification can be realized within the machine-learned model 1100. In particular, the machine-learned model 1100 can perform chewing detection 112 and/or bruxism detection 114 based on sleep detection 118. By referencing sleep detection 118 for performing aspects of chewing detection 112 and/or bruxism detection 114, an overall performance (e.g., accuracy and/or reliability) of chewing detection 112 and/or bruxism detection 114 can be improved. Sleep detection 118 can also assist the machine-learned model 1100 to distinguish between chewing and bruxism as these behaviors may be more likely to occur depending on whether the user 106 is awake or asleep, as further described below.

In an example implementation, the chewing detector 1102 generates the chewing detection indicator 1108 based on the pre-processed signal 710 and the sleep detection indicator 1114. In some cases, chewing may be more likely to occur while the user 106 is awake. As such, the chewing detector 1102 can use the information from the sleep detection indicator 1114 to assist with detecting the presence (or absence) of chewing. For example, the chewing detector 1102 can have a higher level of confidence of detecting the occurrence of chewing if the sleep detection indicator 1114 indicates that the user 106 is awake. Alternatively, the chewing detector 1102 can have a lower level of confidence of detecting the occurrence of chewing if the sleep detection indicator 1114 indicates that the user 106 is asleep. In this example, chewing detection 112 is dependent on sleep detection 118.

Additionally or alternatively, the bruxism detector 1104 generates the bruxism detection indicator 1110 based on the pre-processed signal 710 and a sleep detection indicator 1114, which indicates whether or not the user 106 is sleeping. In some cases, bruxism 202 may be more likely to occur while the user 106 is sleeping. As such, the bruxism detector 1104 can use the information from the sleep detection indicator 1114 to assist with detecting the presence (or absence) of bruxism 202. For example, the bruxism detector 1104 can have a higher level of confidence of detecting bruxism 202 if the sleep detection indicator 1114 indicates that the user 106 is asleep. In this example, bruxism detection 114 is dependent on sleep detection 118.

Still another example implementation of interdependent human behavior detection and/or classification can be realized with the machine-learned model 1100 performing chewing detection 112 based on bruxism detection 114 and/or performing bruxism detection 114 based on chewing detection 112. In these cases, the bruxism detection indicator 1110 can be passed as an input to the chewing detector 1102 and/or the chewing detection indicator 1108 can be passed as an input to the bruxism detector 1104. The interdependency of chewing detection 112 and bruxism detection 114 can enable the machine-learned model 1100 to reduce a likelihood that both bruxism and chewing are detected concurrently. Example operations for generating the sleep detection indicator 1114 are further described with respect to FIG. 12-1.

FIG. 12-1 illustrates an example implementation of a measurement module 520 for performing sleep detection 118 and/or sleep classification 120. In the depicted configuration, the measurement module 520 includes at least one biometric measurement module 1202 and at least one machine-learned model 1204. The biometric measurement module 1202 determines (e.g., measures) two or more biometrics 306 based on the pre-processed signal 710. The machine-learned model 1204 performs sleep detection 118 and/or sleep classification 120 based on the biometrics 306. Although the biometric measurement module 1202 and the machine-learned model 1204 are depicted as separate entities in FIG. 12-1, other implementations of the measurement module 520 can include a machine-learned model 1204 that performs a similar function as the biometric measurement module 1202. The biometric measurement module 1202 can be implemented using data and/or signal processing techniques or can be implemented using another machine-learned model.

The machine-learned model 1204 is implemented using one or more neural networks. A neural network includes a group of connected nodes (e.g., neurons or perceptrons), which are organized into one or more layers. As an example, the machine-learned model 1204 includes a deep neural network with an input layer, an output layer, and one or more hidden layers positioned between the input layer and the output layers. The nodes of the deep neural network can be partially-connected or fully-connected between the layers.

In some implementations, the neural network is a recurrent neural network (e.g., a long short-term memory (LSTM) neural network) with connections between nodes forming a cycle to retain information from a previous portion of an input data sequence for a subsequent portion of the input data sequence. In other cases, the neural network is a feed-forward neural network in which the connections between the nodes do not form a cycle. Additionally or alternatively, the machine-learned model 1204 can include another type of neural network, such as a convolutional neural network. The machine-learned model 1204 can include one or more types of classification models, such as a binary classification model, a multi-class classification model, multi-label classification, and so forth. Other implementations are also possible in which the machine-learned model 1204 includes one or more types of regression models. In this case, the machine-learned model 1204 can output a determined probability, likelihood, or level of confidence associated with the occurrence of the user 106 sleeping.

Through supervised learning, the machine-learned model 1204 is trained to detect and/or classify sleep based on the pre-processed signal 710. In general, the supervised learning can use simulated (e.g., synthetic) data or measured (e.g., real) data for training purposes. The machine-learned model 1204 is trained to generate the sleep detection indicator 1114 and/or a sleep-stage classification 1206 based on the biometrics 306. The machine-learned model 1204 can optionally also perform sleep detection 118 and/or sleep classification 120 using the chewing detection indicator 1108, the bruxism detection indicator 1110, and/or the bruxism type 1112. The chewing detection indicator 1108, the bruxism detection indicator 1110, and the bruxism type 1112 are other example inputs that can be used by the machine-learned model 1204 for sleep detection 118 and/or sleep classification 120. In some implementations, the machine-learned model 1204 can generate the sleep detection indicator 1114 and/or the sleep-stage classification 1206 based on the pre-processed signal 710 or based on a combination of the pre-processed signal 710, the biometrics 306, the chewing detection indicator 1108, the bruxism detection indicator 1110, and/or the bruxism type 1112. By referencing chewing detection 112, bruxism detection 114, and/or bruxism classification 116 for performing aspects of sleep detection 118 and/or sleep classification 120, an overall performance (e.g., accuracy and/or reliability) of sleep detection 118 and/or sleep classification 120 can be improved. For example, if the chewing detection indicator 1108 indicates that the user 106 is chewing, the machine-learned model 1204 can have a higher level of confidence in determining that the user 106 is awake. Additionally or alternatively, if the bruxism detection indicator 1110 indicates that bruxism 202 is occurring, the machine-learned model 1204 can have a higher level of confidence in determining that the user 106 is sleeping.

FIG. 12-2 illustrates an example implementation of the biometric measurement module 1202. In example implementations, the biometric measurement module 1202 can detect the user 106's heart rate 308 and/or respiration rate 310 with an accuracy of 5% or less. The biometric measurement module 1202 can be implemented in various ways depending on which biometrics 306 the biometric measurement module 1202 is designed to measure. The biometric measurement module 1202 can optionally include at least one filter 1210 and can optionally include an autocorrelation module 1212 for measuring the heart rate 308 and/or the respiration rate 310. The biometric measurement module 1202 also includes at least one biometric detector 1214, which measure the one or more biometrics 306 of interest.

The filter 1210 can attenuate frequencies that are outside of a range of interest. For measuring the heart rate 308, for instance, the filter 1210 can pass frequencies associated with a human's heart rate and attenuate frequencies that are outside this range. In this case, the attenuated frequencies can include slower frequencies associated with the respiration rate 310 and/or head movement 318. A similar process can be performed for measuring the respiration rate 310 in which frequencies associated with the heart rate 308 and/or head movement 318 are attenuated.

For measuring the heart rate 308 and/or the respiration rate 310, the autocorrelation module 1212 can generate an autocorrelation 1218 based on the filtered signal 1216. The biometric detector 1214 detects peaks 1220 of the autocorrelation 1218 and measures the time interval between the peaks 1220. This time interval, or period of the autocorrelation 1218, represents the heart rate 308 or the respiration rate 310. At 1222, a graph of an example autocorrelation 1218 is shown having peaks 1220-1 and 1220-2, which can be used to determine the heart rate 308 or the respiration rate 310.

For heart-rate variability, the measurement module 520 may not include the filter 1210 and/or the autocorrelation module 1212. Generally speaking, the biometric detector 1214 can use peak finding estimation to localize the peaks within the pre-processed signal 710. This estimation can be performed across the amplitude 910 and/or phase 912 of the pre-processed signal 710. Example peak finding estimation techniques include Z-score, local maxima, and divide and conquer. The biometric detector 1214 can measure the heart rate variability by calculating a root mean square of successive differences (RMSSD) between each peak (e.g., between each heartbeat).

The biometric detector 1214 can optionally detect occurrences of a dicrotic notch within the pre-processed signal 710. The biometric detector 1214 can determine the blood pressure 312 of the user 106 based on the occurrences of the dicrotic notch.

Other implementations of the measurement module 520 can utilize information determined for sleep detection 118 and/or sleep classification 120 to assist with the detection and/or classification of other human behaviors. Consider for example that the sleep detection indicator 1114 is provided to the chewing detector 1102. With this information, the chewing detector 1102 can have a higher confidence of whether or not chewing is detected. If the sleep detection indicator 1114 indicates that the user 106 is sleeping, the chewing detector 1102 may have higher confidence that the user 106 is not chewing (or is not eating).

In general, the measurement module 520 can be implemented with any combination of the chewing detector 1102, the bruxism detector 1104, the bruxism classifier 1106, and the machine-learned model 1204. In various implementations, the operation of the bruxism detector 1104, the chewing detector 1102, the bruxism classifier 1106, and the machine-learned model 1204 can be arranged in any combination of series and/or parallel configurations. In some cases, a hierarchy or order can be assigned so that certain models are executed prior to others and their information is provided as an input to the other models that are executed later. For example, the bruxism detector 1104 and/or the chewing detector 1102 can execute prior to the machine-learned model 1204 so that the machine-learned model 1204 can utilize the bruxism detection indicator 1110 and/or the chewing detection indicator 1108 to improve the accuracy and reliability of sleep detection 118 and/or sleep classification 120. Other implementations are also possible in which there is a feedback loop so that the output of later-executed models (e.g., the machine-learned model 1204) can be fed back to earlier-executed models (e.g., the bruxism detector 1104, the chewing detector 1102, and/or the bruxism classifier 1106). This allows the earlier-executed models to improve their results. Still other implementations can implement a single machine-learned model in which the bruxism detector 1104, the chewing detector 1102, the bruxism classifier 1106, and the machine-learned model 1204 (or combinations thereof) are combined together.

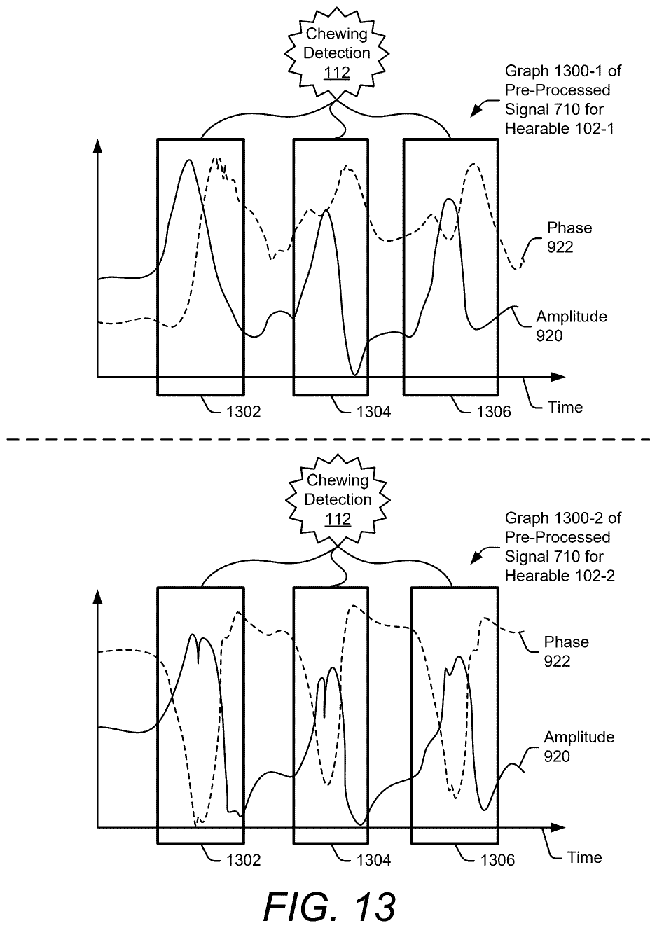

FIGS. 13 to 23 depict example amplitudes 910 and phases 912 of pre-processed signals 710 generated by different hearables 102-1 and 102-2. As shown below, the pressure wave caused by the human behavior can significantly impact the amplitude 910 and/or the phase 912 of the pre-processed signals 710. In some instances, the change in the amplitude 910 and/or the phase 912 can be relative to a previous state or relative to a previous trend in the amplitude 910 and/or the phase 912. The previous state can refer to values of the amplitude 910 and/or the phase 912 during which the human behavior does not occur.

In general, the term “significantly” can mean that the values of the amplitude 910 and/or the phase 912 can change by 10% or more relative to a previous value (e.g., relative to an average of a set of previous values). Additionally or alternatively, a slope of the amplitude 910 and/or the phase 912 can vary significantly. Sometimes the slope of the amplitude 910 and/or the phase 912 can change signs (e.g., from a positive slope to a negative slope, or vice versa). A magnitude of the slope of the amplitude 910 and/or the phase 912 can sometimes change by approximately 10% or more.

In some implementations, the measurement module 520 can perform human behavior detection and/or classification based on the amplitude 910 of the pre-processed signal 710 provided by the hearable 102-1, the phase 912 of the pre-processed signal 710 provided by the hearable 102-1, the amplitude 910 of the pre-processed signal 710 provided by the hearable 102-2, the phase 912 of the pre-processed signal 710 provided by the hearable 102-2, or some combination thereof. Generally speaking, processing a larger quantity of signals and/or tones 704 that are sensitive to the pressure wave caused by the human behavior provides more information to the measurement module 520. This can make it easier for the measurement module 520 to accurately detect and/or classify the human behavior.

FIG. 13 illustrates example pre-processed signals 710 associated with chewing detection 112. Graphs 1300-1 and 1300-2 depict amplitudes 910 and phases 912 of pre-processed signals 710 that are respectively generated by the hearables 102-1 and 102-2. Time is depicted along the horizontal axes of the graphs 1300-1 and 1300-2.

During the time intervals indicated at 1302, 1304, and 1306 the user 106 performs a chewing-type action by moving their jaw and/or tongue. This causes the amplitude 910 and/or the phase 912 of the acoustic receive signal 604 to change significantly relative to a previous state. With audioplethysmography 110, the measurement module 520 can detect and recognize occurrence of the chewing-type action based on the change in the amplitude 910 and/or phase 912 of the pre-processed signals 710 provided by the hearable 102-1 and/or the hearable 102-2.

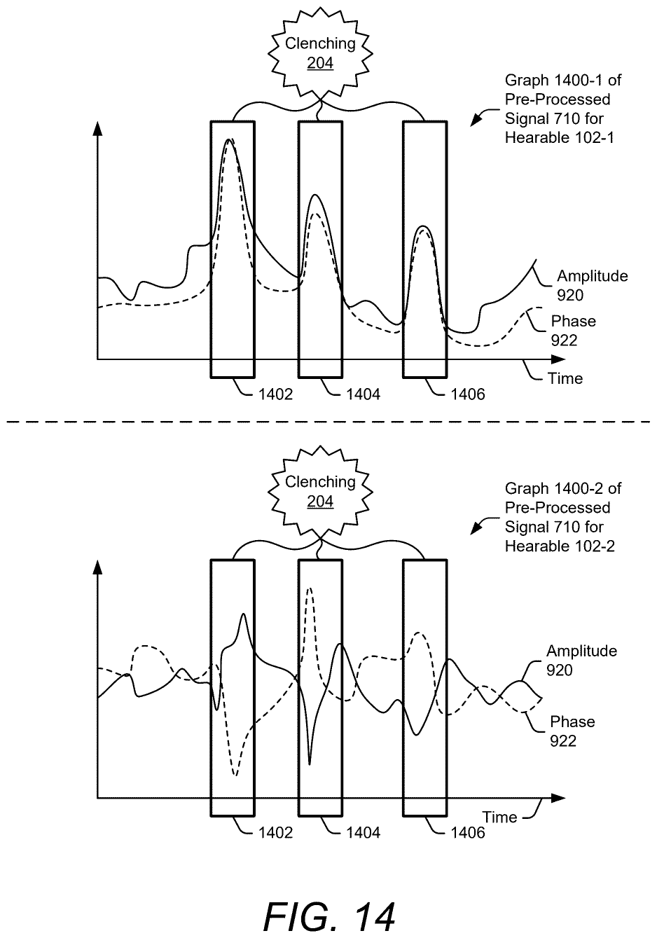

FIG. 14 illustrates example pre-processed signals 710 associated with bruxism detection 114 and/or bruxism classification 116. Graphs 1400-1 and 1400-2 depict amplitudes 910 and phases 912 of pre-processed signals 710 that are respectively generated by the hearables 102-1 and 102-2. Time is depicted along the horizontal axes of the graphs 1400-1 and 1400-2.

During the time intervals indicated at 1402, 1404, and 1406, the user 106 performs a type of bruxism 202 that involves clenching 204. This causes the amplitude 910 and/or the phase 912 of the acoustic receive signal 604 to change significantly relative to a previous state. With audioplethysmography 110, the measurement module 520 can detect and/or classify the clenching 204 based on the change in the amplitude 910 and/or phase 912 of the pre-processed signals 710 provided by the hearable 102-1 and/or the hearable 102-2.

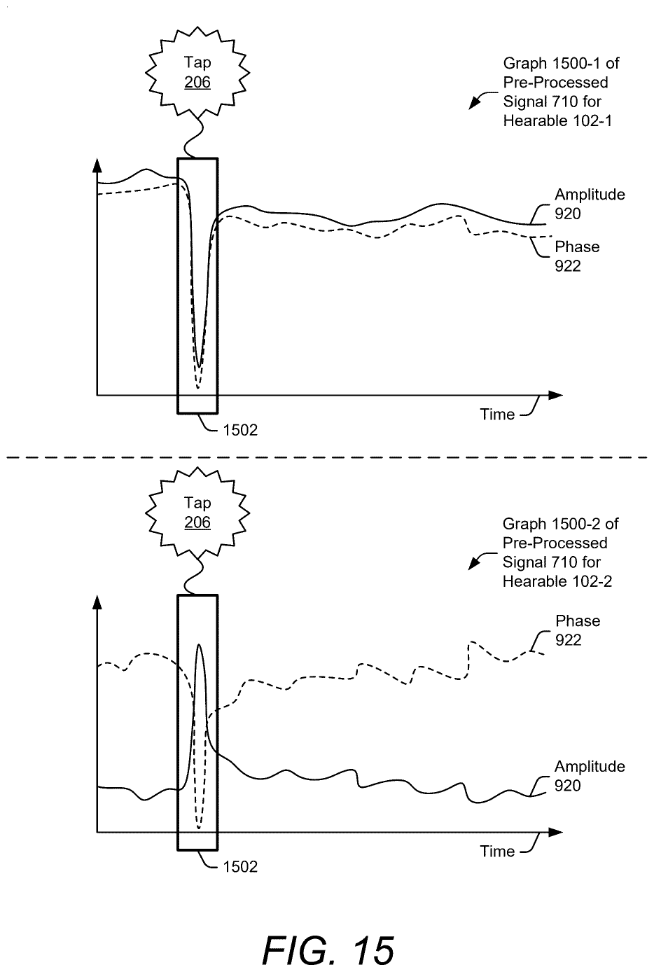

FIG. 15 illustrates example pre-processed signals 710 associated with bruxism detection 114 and/or bruxism classification 116. Graphs 1500-1 and 1500-2 depict amplitudes 910 and phases 912 of pre-processed signals 710 that are respectively generated by the hearables 102-1 and 102-2. Time is depicted along the horizontal axes of the graphs 1500-1 and 1500-2.

During the time interval indicated at 1502, the user 106 performs a type of bruxism 202 that involves a tap 206. This causes the amplitude 910 and/or the phase 912 of the acoustic receive signal 604 to change significantly relative to a previous state. With audioplethysmography 110, the measurement module 520 can detect and/or classify the tap 206 based on the change in the amplitude 910 and/or phase 912 of the pre-processed signals 710 provided by the hearable 102-1 and/or the hearable 102-2.

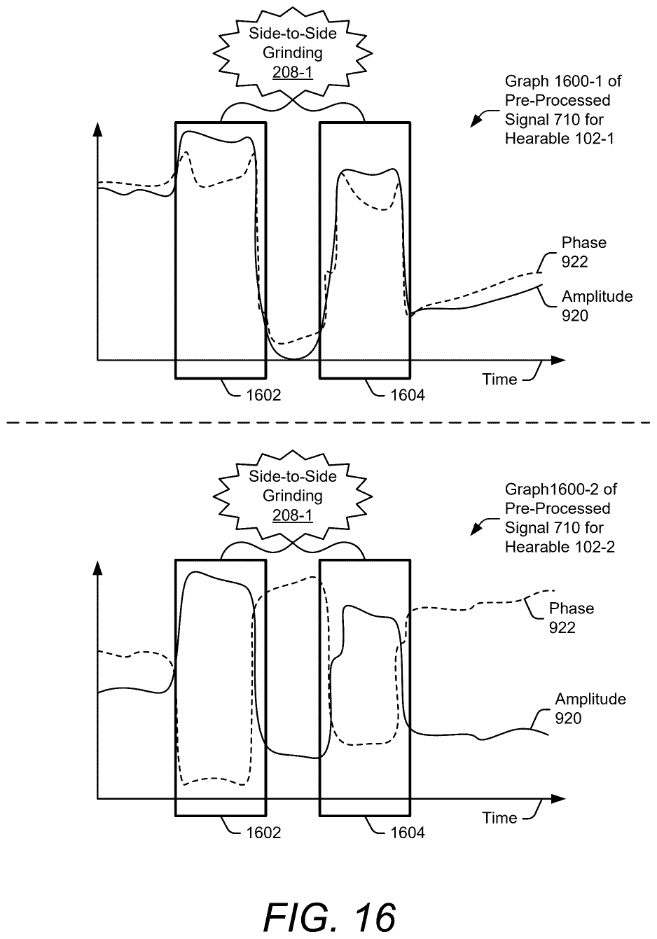

FIG. 16 illustrates example pre-processed signals 710 associated with bruxism detection 114 and/or bruxism classification 116. Graphs 1600-1 and 1600-2 depict amplitudes 910 and phases 912 of pre-processed signals 710 that are respectively generated by the hearables 102-1 and 102-2. Time is depicted along the horizontal axes of the graphs 1600-1 and 1600-2.

During the time intervals indicated at 1602 and 1604, the user 106 performs a type of bruxism 202 that involves side-to-side grinding 208-1. This causes the amplitude 910 and/or the phase 912 of the acoustic receive signal 604 to change significantly relative to a previous state. With audioplethysmography 110, the measurement module 520 can detect and/or classify the side-to-side grinding 208-1 based on the change in the amplitude 910 and/or phase 912 of the pre-processed signals 710 provided by the hearable 102-1 and/or the hearable 102-2.

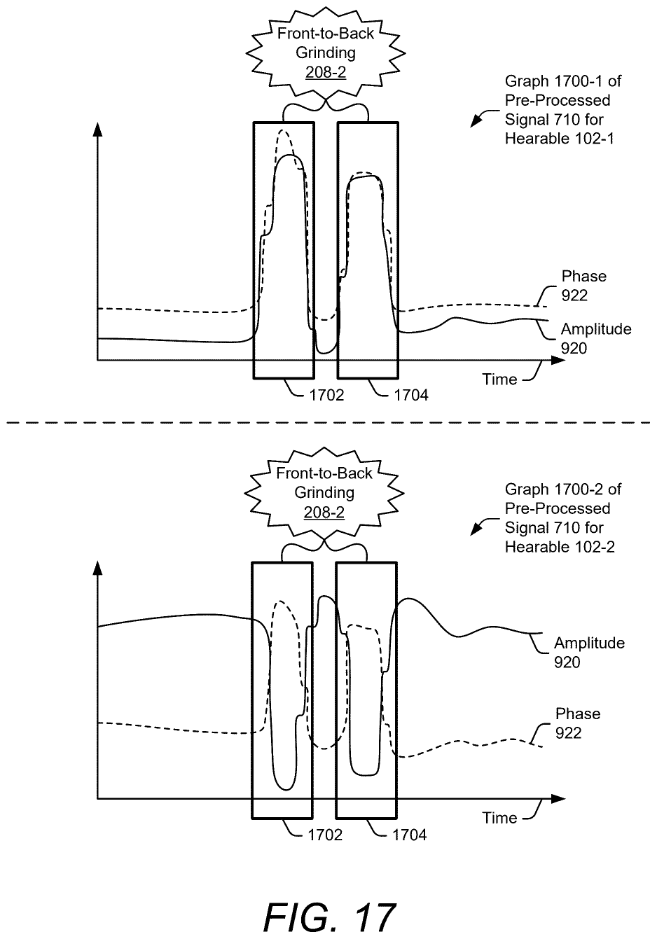

FIG. 17 illustrates example pre-processed signals 710 associated with bruxism detection 114 and/or bruxism classification 116. Graphs 1700-1 and 1700-2 depict amplitudes 910 and phases 912 of pre-processed signals 710 that are respectively generated by the hearables 102-1 and 102-2. Time is depicted along the horizontal axes of the graphs 1700-1 and 1700-2.

During the time intervals indicated at 1702 and 1704, the user 106 performs a type of bruxism 202 that involves front-to-back grinding 208-2. This causes the amplitude 910 and/or the phase 912 of the acoustic receive signal 604 to change significantly relative to a previous state. With audioplethysmography 110, the measurement module 520 can detect and/or classify the front-to-back grinding 208-2 based on the change in the amplitude 910 and/or phase 912 of the pre-processed signals 710 provided by the hearable 102-1 and/or the hearable 102-2.

FIG. 18 illustrates example pre-processed signals 710 across tones 1802-1, 1802-2, and 1802-3. At the top of FIG. 18, graphs 1804-1, 1804-2, and 1804-3 depict an amplitude 910 of the pre-processed signal 710 across the respective tones 1802-1 to 1802-3. The horizontal dimension of the graphs 1804-1 to 1804-3 represent time in seconds, and the vertical dimension of the graphs 1804-1 to 1804-3 represent a normalized amplitude. The amplitudes 910 of the pre-processed signals 710 have peak-to-average ratios 1808-1 to 1808-3 respectively associated with the tones 1802-1 to 1802-3.

In this example, the peak-to-average ratio 1808-2 is higher than the peak-to-average ratio 1808-3, which is higher than the peak-to-average ratio 1808-1. In other words, the tone 1802-2 has the highest peak-to-average ratio 1808-2 across the amplitudes 910. Also, the peak-to-average ratio 1808-2 is greater than a threshold for measuring the heart rate 308.

At the bottom of FIG. 18, graphs 1806-1, 1806-2, and 1806-3 depict a phase 912 of the pre-processed signal 710 across the respective tones 1802-1 to 1802-3. The horizontal dimension of the graphs 1806-1 to 1806-3 represent time in seconds, and the vertical dimension of the graphs 1806-1 to 1806-3 represent a normalized phase. The phase 912 of the pre-processed signal 710 has peak-to-average ratios 1810-1 to 1810-3 respectively associated with the tones 1802-1 to 1802-3.

In this example, the peak-to-average ratio 1810-3 is higher than the peak-to-average ratio 1810-1, which is higher than the peak-to-average ratio 1810-2. In other words, the tone 1802-3 has the highest peak-to-average ratio 1810-3 across the phases 912. Also, the peak-to-average ratio 1810-3 can be greater than a threshold for measuring the heart rate 308 while the peak-to-average ratios 1810-1 and 1810-2 are less than the threshold.

As shown in FIG. 18, cardiac activity (e.g., a heart rate 308) of the user 106 may or may not be detectable within the amplitude 910 or phase 912. For the tone 1802-1, the cardiac activity does not significantly modulate the amplitude 910 or phase 912, which is represented by the relatively low peak-to-average ratios 1808-1 and 1810-1. For the tone 1802-2, the cardiac activity does significantly modulate the amplitude 910 but not the phase 912. For the tone 1802-3, the cardiac activity does not significantly modulate the amplitude 910 but does significantly modulate the phase 912.

With these peak-to-average ratios 1808-1 to 1808-3 and 1810-1 to 1810-3, the frequency selector 1006 can select at least the tone 1802-2 based on the peak-to-average ratio 1808-2 and/or the tone 1802-3 based on the peak-to-average ratio 1810-3 for the measurement procedure. In some cases, the frequency selector 1006 selects only the tone 1802-2, only the tone 1802-3, or both the tones 1802-2 and 1802-3. In situations in which other tones 1802 (not shown) have peak-to-average ratios that are greater than the threshold, these tones 1802 may also be optionally selected by the frequency selector 1006.

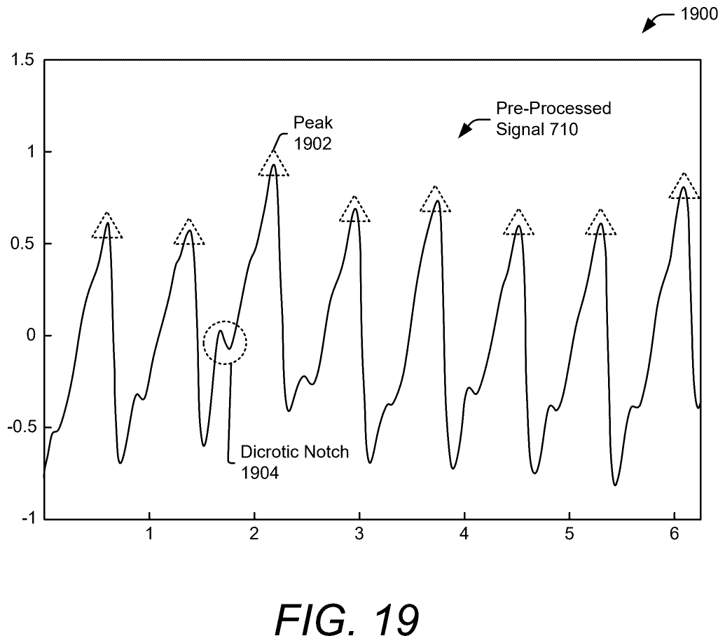

FIG. 19 illustrates an example graph 1900 of an amplitude 910 of the pre-processed signal 710 for detecting heart rate variability or blood pressure using the hearable 102. A horizontal dimension of the graph 1900 represents time in seconds, and a vertical dimension of the graph 1900 represents a normalized amplitude. The pre-processed signal 710 has peaks 1902, which are identified using triangles. The pre-processed signal 710 also has dicrotic notches 1904, an example of which is circled in FIG. 19. Each peak 1902 is associated with a heartbeat of the user 106, and can be identified by the biometric measurement module 1202 to measure the heart rate variability. Each dicrotic notch 1904 can be identified by the biometric measurement module 1202 to measure the blood pressure 312.

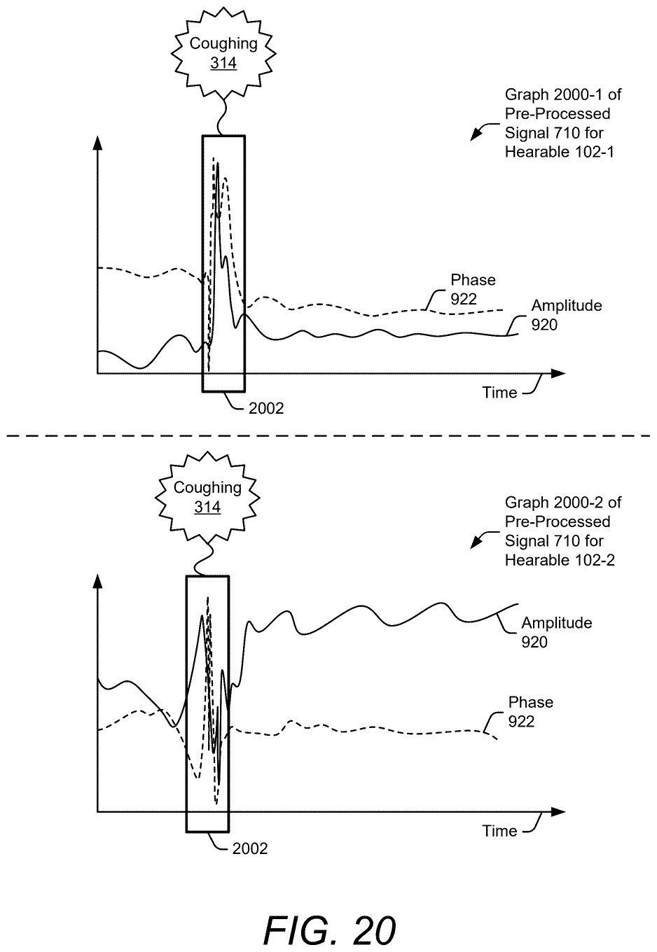

FIG. 20 illustrates example pre-processed signals 710 associated with sleep classification 120. Graphs 2000-1 and 2000-2 depict amplitudes 910 and phases 912 of pre-processed signals 710 that are respectively generated by the hearables 102-1 and 102-2. Time is depicted along the horizontal axes of the graphs 2000-1 and 2000-2.

During the time interval indicated at 2002, the user 106 is coughing 314. This causes the amplitude 910 and/or the phase 912 of the acoustic receive signal 604 to change significantly relative to a previous state. With audioplethysmography 110, the measurement module 520 can detect and recognize the coughing 314 based on the change in the amplitude 910 and/or phase 912 of the pre-processed signals 710 provided by the hearable 102-1 and/or the hearable 102-2. The occurrence of the coughing 314 can be used to classify a current sleep stage in which the user 106 is coughing 314. Coughing 314, for instance, can indicate that the user 106 is currently sleeping in the first or second stage 304-1 or 304-2.

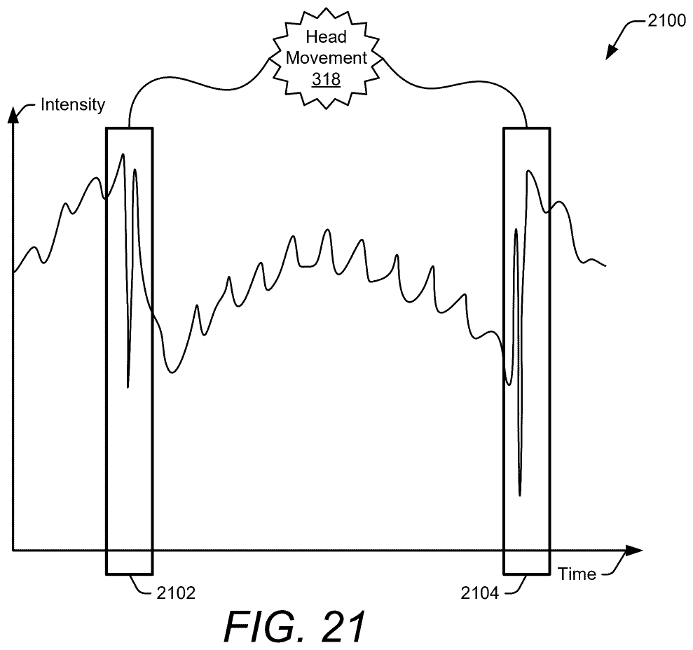

FIG. 21 illustrates an example pre-processed signal 701 associated with sleep detection 118 and/or sleep classification 120. Graph 2100 depicts an amplitude 910 of the pre-processed signal 710 that is generated by the hearable 102-1 or 102-2. Time is depicted along the horizontal axes of the graph 2100.

During the time intervals indicated at 2102 and 2104, the user 106 moves their head (e.g., head movement 318 occurs). This causes the amplitude 910 of the acoustic receive signal 604 to change significantly relative to a previous state. With audioplethysmography 110, the measurement module 520 can detect and recognize occurrence of the head movement 318 based on the change in the amplitude 910 of the pre-processed signal 710 provided by the hearable 102-1 and/or 102-2. Although not explicitly shown, the head movement 318 can also be detected and/or recognized based on a change in the phase 912 of the pre-processed signal 710.

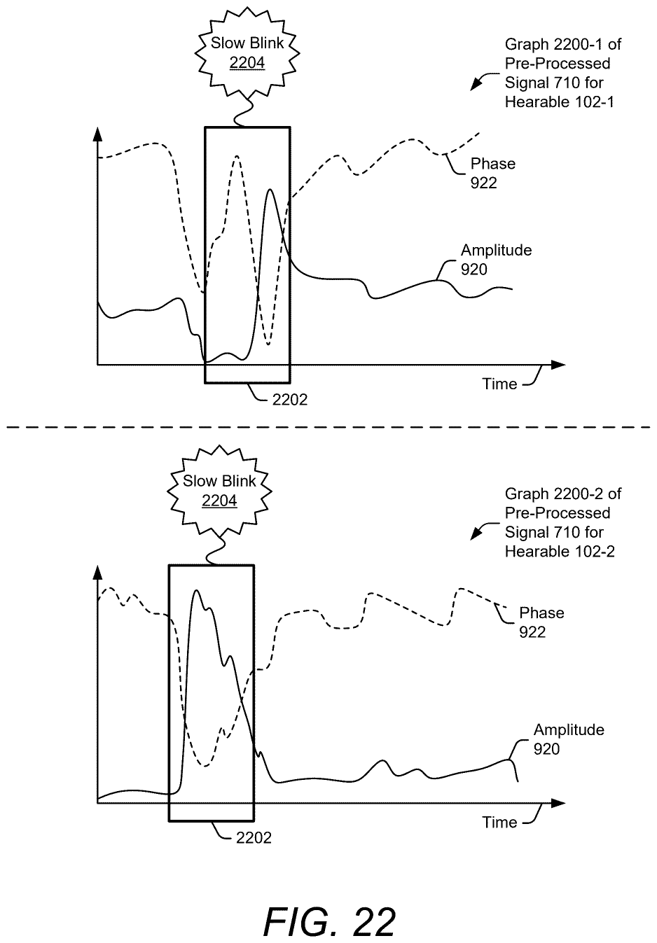

FIG. 22 illustrates example pre-processed signals 710 associated with sleep detection 118 and/or sleep classification 120. Graphs 2200-1 and 2200-2 depict amplitudes 910 and phases 912 of pre-processed signals 710 that are respectively generated by the hearables 102-1 and 102-2. Time is depicted along the horizontal axes of the graphs 2200-1 and 2200-2.

During the time interval indicated at 2202, the user 106 slowly blinks 2204. This causes the amplitude 910 and/or the phase 912 of the acoustic receive signal 604 to change significantly relative to a previous state. With audioplethysmography 110, the measurement module 520 can detect and recognize occurrence of the slow blink 2204 based on the change in the amplitude 910 and/or phase 912 of the pre-processed signals 710 provided by the hearable 102-1 and/or the hearable 102-2.

The slow blink 2204 represents a type of eye movement 320, which can be used for sleep detection 118 and/or sleep classification 120. Occurrence of the slow blink 2204 can indicate that the user 106 is in the process of falling asleep, for instance. The slow blink 2204 can also be associated with the first and/or second stages 304-1 and 304-2 of sleep.

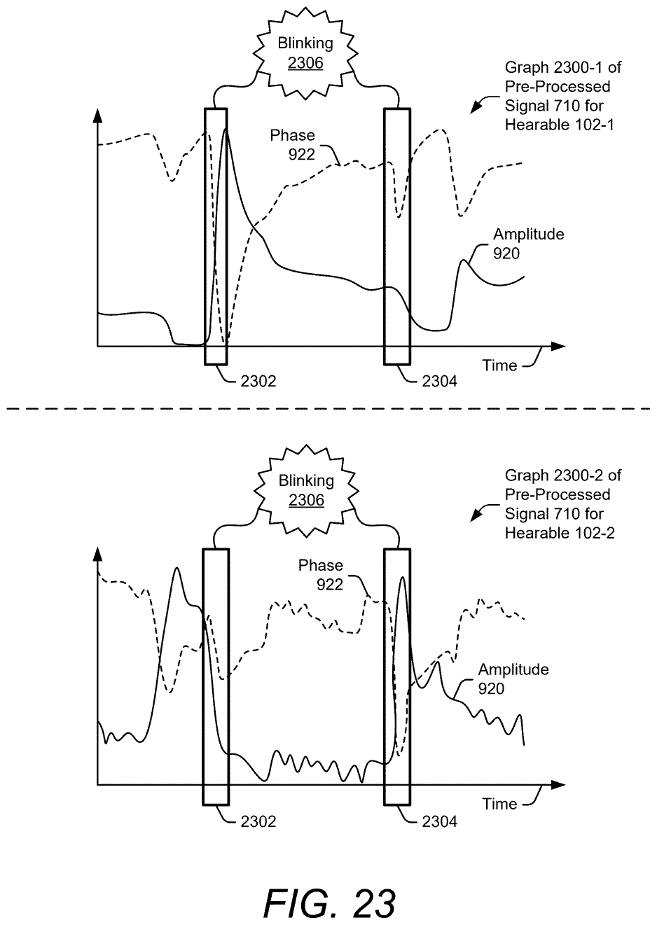

FIG. 23 illustrates example pre-processed signals 710 associated with sleep detection 118 and/or sleep classification 120. Graphs 2300-1 and 2300-2 depict amplitudes 910 and phases 912 of pre-processed signals 710 that are respectively generated by the hearables 102-1 and 102-2. Time is depicted along the horizontal axes of the graphs 2300-1 and 2300-2.

During the time intervals indicated at 2302 and 2304, the user 106 blinks 2306. This causes the amplitude 910 and/or the phase 912 of the acoustic receive signal 604 to change significantly relative to a previous state. With audioplethysmography 110, the measurement module 520 can detect and recognize the blinking 2306 based on the change in the amplitude 910 and/or phase 912 of the pre-processed signals 710 provided by the hearable 102-1 and/or the hearable 102-2.

The blinking 2306 represents a type of eye movement 320, which can be used for sleep detection 118 and/or sleep classification 120. Occurrence of the blinking 2306 can indicate that the user 106 is awake, for instance. If the user 106 is asleep, the blinking 2306 can be associated with the first and/or second stages 304-1 and 304-2 of sleep.

The signals depicted within the graphs of FIGS. 13 to 23 are associated with a particular tone 704. In some cases, multiple tones 704 of the acoustic receive signal 604 are used to detect and/or classify a human behavior. The signals depicted in FIGS. 13 to 23 generally represent smoothed data. Signals that are generated using audioplethysmography 110 can have additional noise that is not depicted in the graphs of FIGS. 13 to 23 for simplicity and clarity. In most of the signals depicted in FIGS. 13 to 23, both the amplitude 910 and the phase 912 are impacted by the human behavior and can be used to detect and/or classify the human behavior. Sometimes, however, only one of the amplitude 910 or the phase 912 are impacted by the human behavior. However, human behavior detection and/or classification can still be performed in this instance. Also, sometimes only one of the hearables 102-1 or 102-2 are impacted by the human behavior. If the amplitude 910 and/or the phase 912 of a pre-processed signal 710 does not show a significant impact based on the human behavior, the measurement module 520 can rely on other tones 704 or other pre-processed signals 710 (e.g., provided by a different hearable 102) to perform human behavior detection and/or classification.

Although the example pre-processed signals 710 shown in FIGS. 13 to 23 are associated with a single human behavior, other pre-processed signals 710 can include amplitude and/or phase variations that are indicative of multiple human behaviors. The measurement module 420 can employ various signal processing and/or machine-learning techniques to separate out the features of interest within the pre-processed signals 710 for a particular human behavior. Or the measurement module 420 can be designed and/or trained to recognize multiple human behaviors at once based on the amplitude and/or phase characteristics of the pre-processed signal 710.

Consider a first example in which the pre-processed signal 710 includes the variations described with respect to FIG. 14 for clenching 204 and also includes variations associated with the user 106's heart rate 308. In this case, the measurement module 420 can analyze the pre-processed signal 710 to detect the occurrence of clenching 204 based on the substantial variation in the amplitude 902 and/or phase 922 as well as the frequency of this variation. At the same time, the measurement module 420 can further process the pre-processed signal 710 to measure the user 106's heart rate 308 and determine that the heart rate 308 indicates that the user 106 is asleep. In this case, the measurement module 420 can detect the presence of bruxism 202 and detect that the user 106 is asleep.

Consider a second example in which the pre-processed signal 710 does not include the variations described with respect to FIGS. 13-17 but includes the variations that enable the user 106's heart rate 308 to be measured. In this case, the measurement module 420 can detect the absence of bruxism 202 and chewing and detect that the user 106 is asleep based on the heart rate 308. Generally speaking, the pre-processed signal 710 can be processed in a manner that enables the occurrence (or absence) of any of the described human behaviors as well as any of the described biometrics 306 to be determined and/or measured.

Aspects of human behavior detection and/or classification can be performed using one hearable 102 (e.g., the hearable 102-1 or 102-2) or multiple hearables 102 (e.g., the hearables 102-1 and 102-2). Performing human behavior detection and/or classification using multiple hearables 102 can improve a confidence level for detecting and/or correctly classifying the human behavior. In general, the hearable 102 can detect and/or classify a human behavior by analyzing changes in the amplitude 910 of the acoustic receive signal 604, changes in the phase 912 of the acoustic receive signal 604, or changes in both the amplitude 910 and phase 912 of the acoustic receive signal 604.

### Example Methods

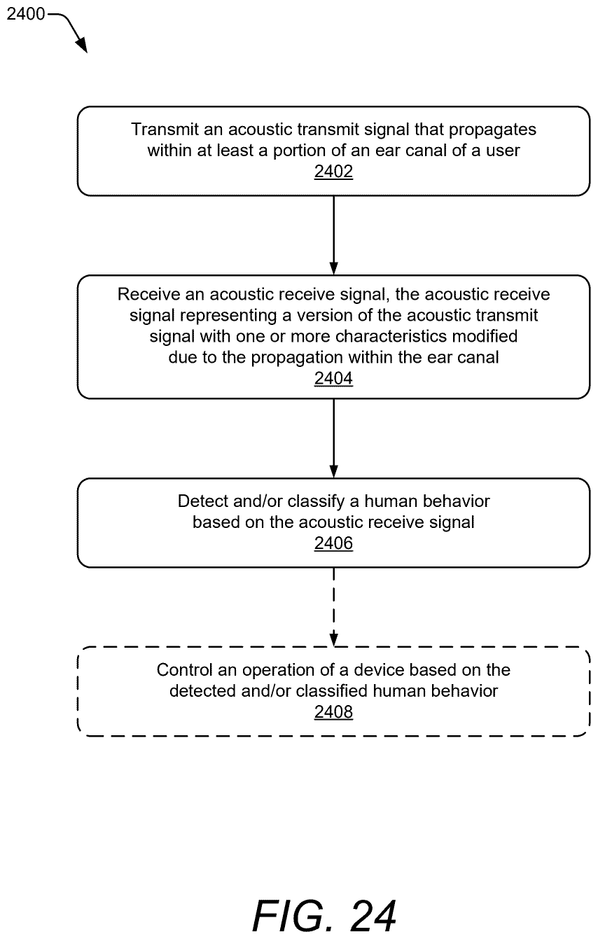

FIGS. 24, 25, 26, 27, 28, 29, and 30 depict example methods 2400, 2500, 2600, 2700, 2800, 2900, and 3000 for implementing aspects of human behavior detection and/or classification using active acoustic sensing. Methods 2400-3000 are shown as sets of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to the environments 100, 200-1, 200-2, 200-3 of FIGS. 1-1, 2-1, and 2-2, and entities detailed in FIGS. 4, 5, 11, and 12-1, reference to which is made for example only. The techniques are not limited to performance by one entity or multiple entities operating on one device.

At 2402 in FIG. 24, an acoustic transmit signal that propagates within at least a portion of an ear canal of a user is transmitted. For example, the hearable 102 transmits the acoustic transmit signal 602, which propagates within at least a portion of the ear canal 124 of the user 106, as shown in FIG. 6. The acoustic transmit signal 602 can include multiple tones 704-1 to 704-N to improve performance for detecting and/or classifying human behavior.

At 2404, an acoustic receive signal is received. The acoustic receive signal represents a version of the acoustic transmit signal with one or more characteristics modified due to the propagation within the ear canal. For example, the hearable 102 receives the acoustic receive signal 604, as shown in FIG. 6. The acoustic receive signal 604 represents a version of the acoustic transmit signal 602 with one or more characteristics (or waveform characteristics) modified due to the propagation within the ear canal 124. Example characteristics include amplitude, frequency, and/or phase. The hearable 102 that receives the acoustic receive signal 604 can be a same hearable 102 that transmitted the acoustic transmit signal 602 (e.g., the hearable 102-1 or 102-2 in FIG. 6), or another hearable 102 that did not transmit the acoustic transmit signal 602 (e.g., the hearable 102-2 in FIG. 6).

At 2406, a human behavior is detected and/or classified based on the acoustic receive signal. For example, the measurement module 520 detects and/or classifies the human behavior. The human behavior can include chewing (or eating), bruxism, and/or sleeping. In some implementations, the measurement module 520 includes at least one machine-learned model that is trained using supervised learning to detect and/or classify the human behavior.

At 2408, an operation of a device is controlled based on the detected and/or classified human behavior. For example, an operation of the hearable 102 and/or the computing device 104 is controlled based on the detected and/or classified human behavior. Various controls can include adjusting a volume, playing a particular type of audio content, sounding an alarm, changing between operational modes (e.g., changing between a high-power mode and a low-power mode), logging data for the user 106, providing data to another device, and so forth.

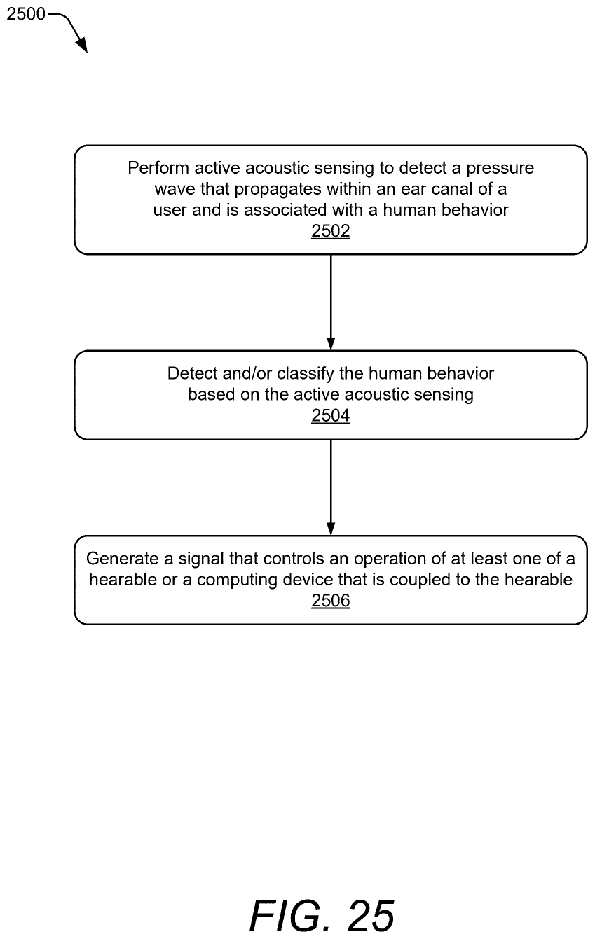

At 2502 in FIG. 25, active acoustic sensing is performed to detect a pressure wave that propagates to an ear canal of a user and is associated with a human behavior. For example, the hearable 102 performs active acoustic sensing to detect the pressure wave that propagates to the ear canal 124 of the user 106 and is associated with a human behavior, such as chewing (or eating), bruxism, or sleeping. More specifically, the hearable 102 transmits and receives an acoustic signal during the first time period. The acoustic signal propagates within at least a portion of the ear canal 124 of the user 106. The received acoustic signal (e.g., the acoustic receive signal 604) represents a version of the transmitted acoustic signal (e.g., the acoustic transmit signal 602) with one or more characteristics (e.g., amplitude 910 and/or phase 912) modified based on the propagation within the ear canal 124 and based on a human behavior that occurs during at least a portion of the first time period.

At 2504, the human behavior is detected and/or classified based on the active acoustic sensing. For example, the measurement module 520 performs chewing detection 112, bruxism detection 114, bruxism classification 116, sleep detection 118, and/or sleep classification 120 based on the active acoustic sensing (e.g., based on the version of the acoustic receive signal 604, such as the pre-processed signal 710).

At 2506, a signal that controls an operation of at least one of a hearable or a computing device that is coupled to the hearable is generated. For example the measurement module 520 generates a control signal to control an operation of the hearable 102 and/or the computing device 104. The control signal can be represented as part of the audioplethysmography data 712 in FIG. 7.

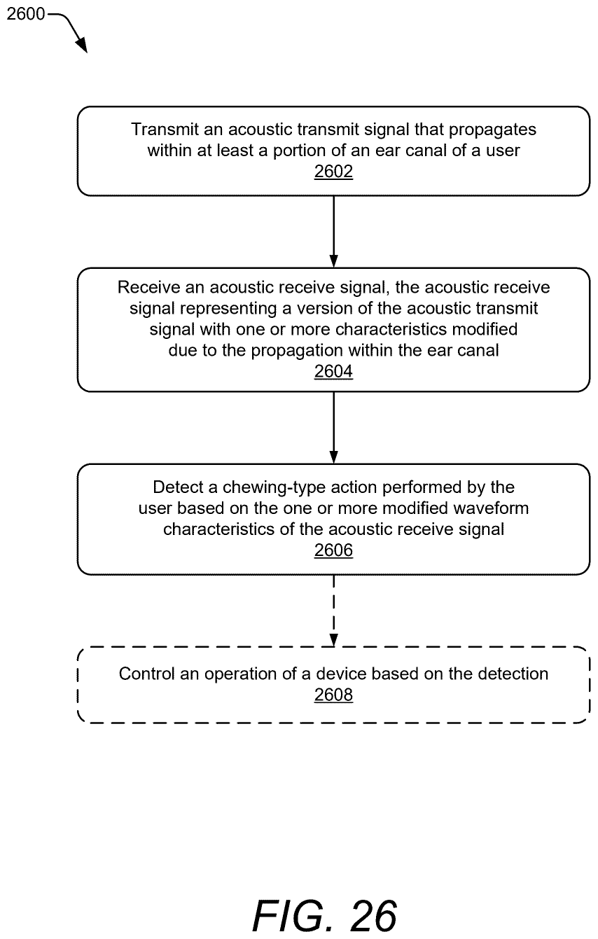

At 2602 in FIG. 26, an acoustic transmit signal that propagates within at least a portion of an ear canal of a user is transmitted. For example, the hearable 102 transmits the acoustic transmit signal 602, which propagates within at least a portion of the ear canal 124 of the user 106, as shown in FIG. 6. The acoustic transmit signal 602 can include multiple tones 704-1 to 704-N to improve performance for detecting and/or classifying human behavior.

At 2604, an acoustic receive signal is received. The acoustic receive signal represents a version of the acoustic transmit signal with one or more characteristics modified due to the propagation within the ear canal. For example, the hearable 102 receives the acoustic receive signal 604, as shown in FIG. 6. The acoustic receive signal 604 represents a version of the acoustic transmit signal 602 with one or more characteristics (e.g., amplitude, frequency, and/or phase) modified due to the propagation within the ear canal 124. The hearable 102 that receives the acoustic receive signal 604 can be a same hearable 102 that transmitted the acoustic transmit signal 602 (e.g., the hearable 102-1 or 102-2 in FIG. 6), or another hearable 102 that did not transmit the acoustic transmit signal 602 (e.g., the hearable 102-2 in FIG. 6).

At 2606, a chewing-type action performed by the user is detected based on the one or more modified characteristics of the acoustic receive signal. For example, the measurement module 520 detects the chewing-type action that is performed by the user 106 during at least a portion of the time in which the acoustic transmit signal 602 is transmitted and/or the acoustic receive signal 602 is received.

At 2608, an operation of a device is controlled based on the detection. For example, an operation of the hearable 102 and/or the computing device 104 is controlled based on the detection. Example controls can include logging a time associated with the chewing-type action, determining a duration of the chewing-type action, estimating an amount of calories that are consumed based on the duration of the chewing-type action, determining if the chewing-type action occurs outside time windows associated with meals, sounding an alarm or sending a notification if the chewing-type action occurs outside of the time windows associated with meals, counting the quantity of chewing-type actions, playing audible content to encourage the user 106 to continue performing the chewing-type action, pausing the audible content if the user 106 stops performing the chewing-type action, providing recommendations for improving eating habits (e.g., recommending eating at a particular time or eating a particular food to facilitate longer periods of intermittent fasting), evaluating results associated with implementing a recommendation, and so forth.

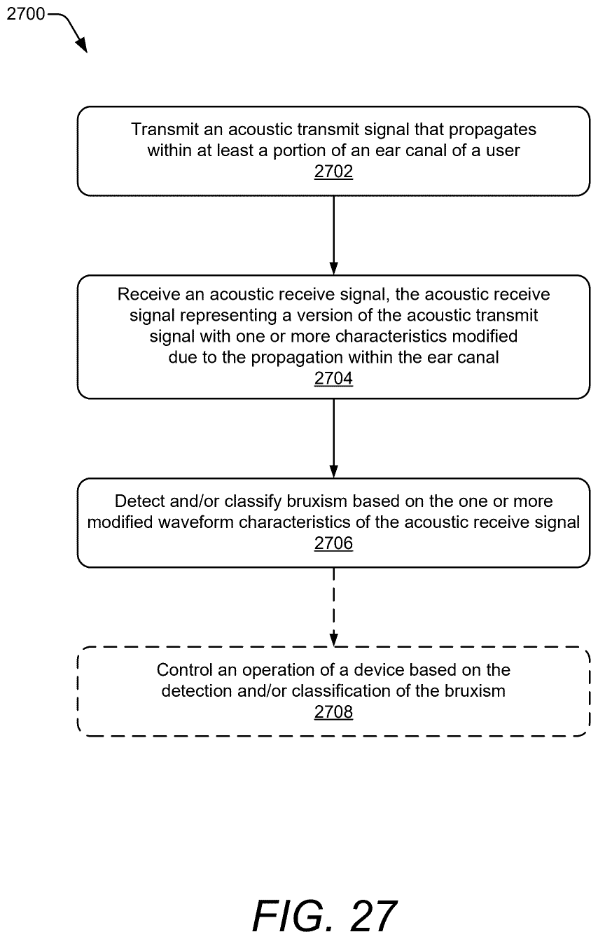

At 2702 in FIG. 27, an acoustic transmit signal that propagates within at least a portion of an ear canal of a user is transmitted. For example, the hearable 102 transmits the acoustic transmit signal 602, which propagates within at least a portion of the ear canal 124 of the user 106, as shown in FIG. 6. The acoustic transmit signal 602 can include multiple tones 704-1 to 704-N to improve performance for detecting and/or classifying human behavior.

At 2704, an acoustic receive signal is received. The acoustic receive signal represents a version of the acoustic transmit signal with one or more characteristics modified due to the propagation within the ear canal. For example, the hearable 102 receives the acoustic receive signal 604, as shown in FIG. 6. The acoustic receive signal 604 represents a version of the acoustic transmit signal 602 with one or more characteristics (e.g., amplitude, frequency, and/or phase) modified due to the propagation within the ear canal 124. The hearable 102 that receives the acoustic receive signal 604 can be a same hearable 102 that transmitted the acoustic transmit signal 602 (e.g., the hearable 102-1 or 102-2 in FIG. 6), or another hearable 102 that did not transmit the acoustic transmit signal 602 (e.g., the hearable 102-2 in FIG. 6).

At 2706, bruxism is detected and/or classified based on the one or more modified characteristics of the acoustic receive signal. For example, the measurement module 520 performs bruxism detection 114 and/or bruxism classification 116 to detect and/or classify bruxism 202 based on the pre-processed signal 710. The bruxism 202 can occur during at least a portion of time in which the acoustic transmit signal 602 is transmitted and/or the acoustic receive signal 602 is received.

At 2708, an operation of a device is controlled based on the detection and/or classification of the bruxism. For example, an operation of the hearable 102 and/or the computing device 104 is controlled based on the detection and/or classification of the bruxism 202. Example controls can include logging a time associated with the bruxism 202, logging a type of bruxism 202 (e.g., clenching 204, tapping 206, and/or grinding 208), determining a duration of the bruxism 202, sounding an alarm or sending a notification responsive to detecting the bruxism 202, determining an emotional state of the user 106 based on the occurrence of bruxism 202, estimating the user 106's level of stress based on the occurrence of bruxism 202, analyzing sleep quality based on the occurrence of bruxism 202, providing recommendations for reducing the occurrence of bruxism 202 (e.g., recommending meditation or playing relaxing music to reduce stress), evaluating results associated with implementing a recommendation, and so forth.

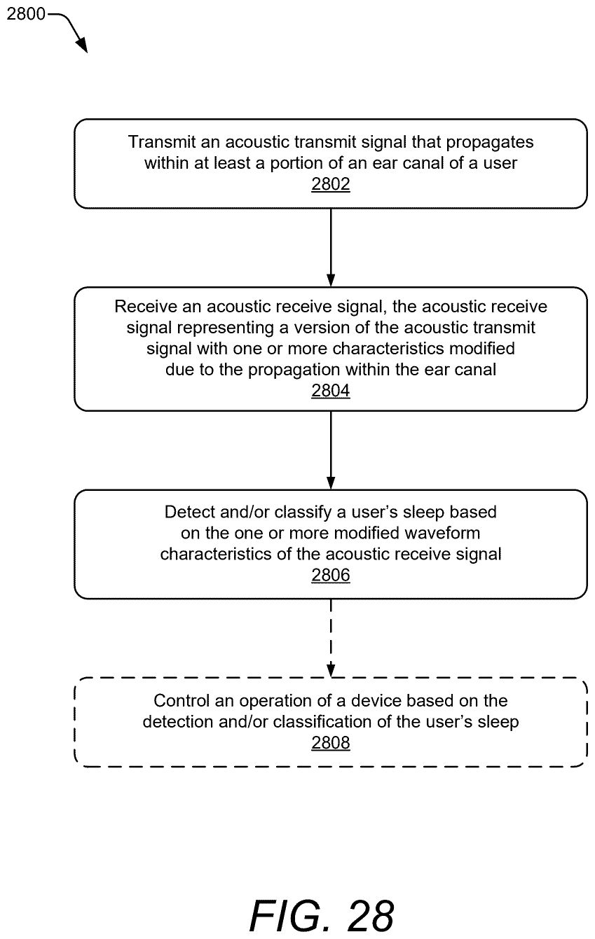

At 2802 in FIG. 28, an acoustic transmit signal that propagates within at least a portion of an ear canal of a user is transmitted. For example, the hearable 102 transmits the acoustic transmit signal 602, which propagates within at least a portion of the ear canal 124 of the user 106, as shown in FIG. 6. The acoustic transmit signal 602 can include multiple tones 704-1 to 704-N to improve performance for detecting and/or classifying human behavior.

At 2804, an acoustic receive signal is received. The acoustic receive signal represents a version of the acoustic transmit signal with one or more characteristics modified due to the propagation within the ear canal. For example, the hearable 102 receives the acoustic receive signal 604, as shown in FIG. 6. The acoustic receive signal 604 represents a version of the acoustic transmit signal 602 with one or more characteristics (e.g., amplitude, frequency, and/or phase) modified due to the propagation within the ear canal 124. The hearable 102 that receives the acoustic receive signal 604 can be a same hearable 102 that transmitted the acoustic transmit signal 602 (e.g., the hearable 102-1 or 102-2 in FIG. 6), or another hearable 102 that did not transmit the acoustic transmit signal 602 (e.g., the hearable 102-2 in FIG. 6).

At 2806, a user's sleep is detected and/or classified based on the one or more modified characteristics of the acoustic receive signal. For example, the measurement module 520 performs sleep detection 118 and/or sleep classification 120 to detect and/or classify the user 106's sleep based on the pre-processed signal 710. The user 106 can be asleep during at least a portion of the time in which the acoustic transmit signal 602 is transmitted and/or the acoustic receive signal 602 is received.

At 2808, an operation of a device is controlled based on the detection and/or classification of the bruxism. For example, an operation of the hearable 102 and/or the computing device 104 is controlled based on the detection and/or classification of the user 106's sleep. Example controls can include switching operational modes (e.g., changing between a normal mode and a sleep mode or changing between a high-power mode and a low-power mode), logging a time in which the user 106 falls asleep, logging a duration of the user 106's sleep, logging a time and/or duration associated with each stage 304 of sleep, performing sleep quality analysis, adjusting a wake-up alarm based on the determined sleep quality or a current sleep stage 304, pausing audible content after the user 106 falls asleep, providing recommendations for improving sleep (e.g., recommending going to bed at a particular time, recommending meditation prior to bedtime, or recommending certain music or white noise to assist with deep sleep), evaluating results associated with implementing a recommendation, and so forth.

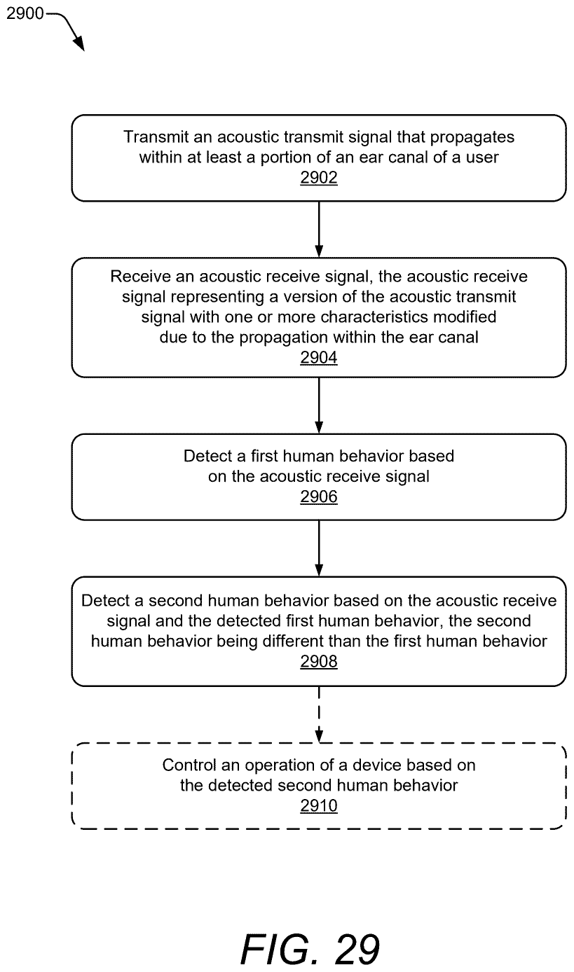

At 2902 in FIG. 29, an acoustic transmit signal that propagates within at least a portion of an ear canal of a user is transmitted. For example, the hearable 102 transmits the acoustic transmit signal 602, which propagates within at least a portion of the ear canal 124 of the user 106, as shown in FIG. 6. The acoustic transmit signal 602 can include multiple tones 704-1 to 704-N to improve performance for detecting and/or classifying human behavior.

At 2904, an acoustic receive signal is received. The acoustic receive signal represents a version of the acoustic transmit signal with one or more characteristics modified due to the propagation within the ear canal. For example, the hearable 102 receives the acoustic receive signal 604, as shown in FIG. 6. The acoustic receive signal 604 represents a version of the acoustic transmit signal 602 with one or more characteristics (e.g., amplitude, frequency, and/or phase) modified due to the propagation within the ear canal 124. The hearable 102 that receives the acoustic receive signal 604 can be a same hearable 102 that transmitted the acoustic transmit signal 602 (e.g., the hearable 102-1 or 102-2 in FIG. 6), or another hearable 102 that did not transmit the acoustic transmit signal 602 (e.g., the hearable 102-2 in FIG. 6).

At 2906, a first human behavior is detected based on the acoustic receive signal. For example, the measurement module 520 detects a first human behavior based on the acoustic receive signal 604 (or based on a signal derived from the acoustic receive signal 604). The first human behavior can include chewing (or eating), bruxism, and/or sleeping. In some implementations, the measurement module 520 includes at least one machine-learned model that is trained using supervised learning to detect and/or classify the first human behavior.

At 2908, a second human behavior is detected based on the acoustic receive signal and the detected first human behavior. The second human behavior is different than the first human behavior. For example, the measurement module 520 detects a second human behavior based on the acoustic receive signal 604 (or based on a signal derived from the acoustic receive signal 604) and the first human behavior. The second human behavior is different than the first human behavior. The second human behavior can include chewing (or eating), bruxism, and/or sleeping.

By detecting the second human behavior based on the first human behavior, the hearable 102 performs an aspect of interdependent human behavior detection and/or classification using active acoustic sensing, which can improve a performance of the hearable 102 for detecting the second human behavior. Aspects of interdependent human behavior detection and/or classification can also be performed with respect to detecting an absence of the first human behavior and detecting an occurrence or an absence of a third human behavior based on the acoustic receive signal 604 and the absence of the first human behavior.

At 2910, an operation of a device is controlled based on the detected second human behavior. For example, an operation of the hearable 102 and/or the computing device 104 is controlled based on the detected second human behavior. Various controls can include adjusting a volume, playing a particular type of audio content, sounding an alarm, changing between operational modes (e.g., changing between a high-power mode and a low-power mode), logging data for the user 106, providing data to another device, and so forth.

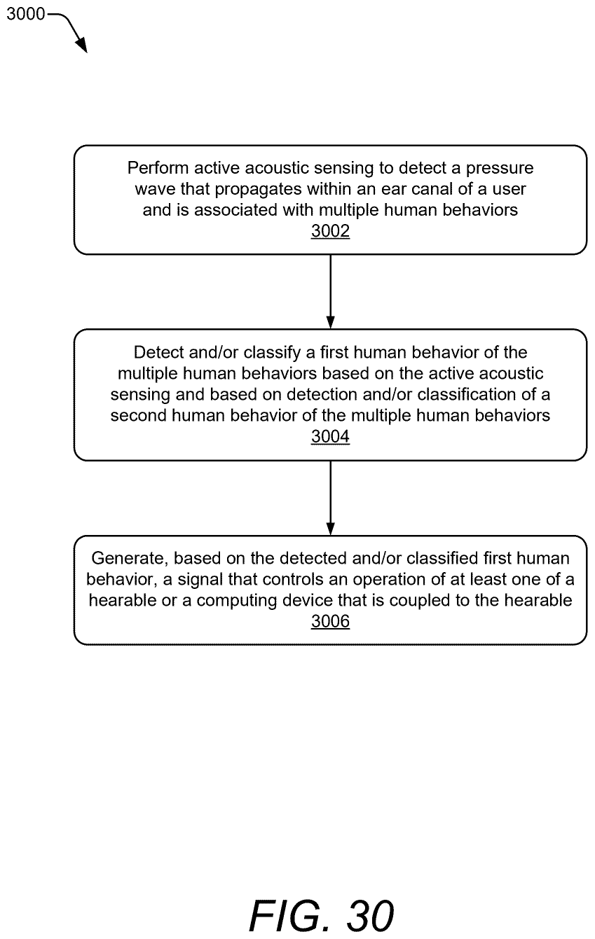

At 3002 in FIG. 30, active acoustic sensing is performed to detect a pressure wave that propagates to an ear canal of a user and is associated with multiple human behaviors. For example, the hearable 102 performs active acoustic sensing to detect the pressure wave that propagates to the ear canal 124 of the user 106 and is associated with multiple human behaviors, such as chewing (or eating), bruxism, or sleeping. More specifically, the hearable 102 transmits and receives an acoustic signal during the first time period. The acoustic signal propagates within at least a portion of the ear canal 124 of the user 106. The received acoustic signal (e.g., the acoustic receive signal 604) represents a version of the transmitted acoustic signal (e.g., the acoustic transmit signal 602) with one or more characteristics (e.g., amplitude 910 and/or phase 912) modified based on the propagation within the ear canal 124 and based on multiple human behaviors that occur (or do not occur) during at least a portion of the first time period.

At 3004, a first human behavior of the multiple human behaviors is detected and/or classified based on the active acoustic sensing and based on detection and/or classification of a second human behavior of the multiple human behaviors. For example, the measurement module 520 detects and/or classifies a first human behavior by performing chewing detection 112, bruxism detection 114, bruxism classification 116, sleep detection 118, and/or sleep classification 120. The detection and/or classification of the first human behavior is based on the active acoustic sensing (e.g., based on the version of the acoustic receive signal 604, such as the pre-processed signal 710) and based on detection and/or classification of a second human behavior of the multiple human behaviors. The second human behavior is different than the first human behavior. The first human behavior and the second human behavior can include different behaviors of the following list: chewing (or eating), bruxism, or sleeping. In this manner, the hearable 102 performs an aspect of interdependent human behavior detection and/or recognition.

At 3006, a signal that controls an operation of at least one of a hearable or a computing device that is coupled to the hearable is generated based on the detected and/or classified first human behavior. For example the measurement module 520 generates a control signal to control an operation of the hearable 102 and/or the computing device 104 based on the detected and/or classified first behavior. The control signal can be represented as part of the audioplethysmography data 712 in FIG. 7.

### Example Computing System

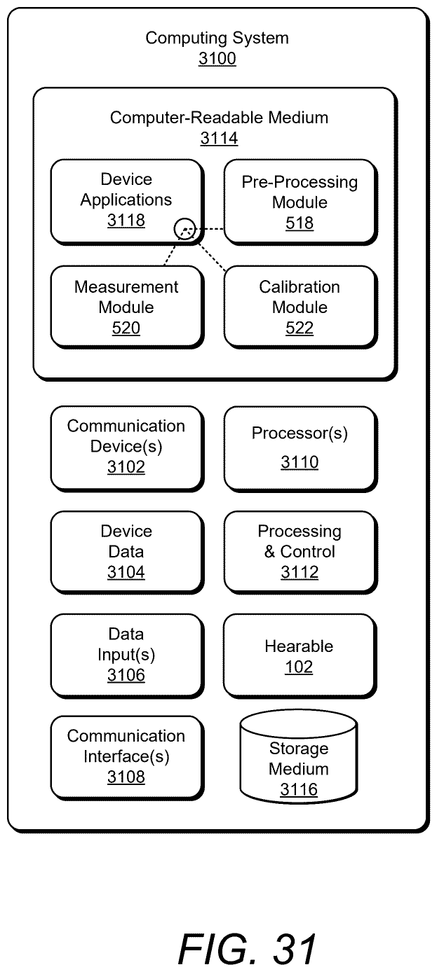

FIG. 31 illustrates various components of an example computing system 3100 that can be implemented as any type of client, server, and/or computing device as described with reference to the previous FIGS. 4 and 5 to implement aspects of interdependent human behavior detection and/or classification using active acoustic sensing.

The computing system 3100 includes communication devices 3102 that enable wired and/or wireless communication of device data 3104 (e.g., received data, data that is being received, data scheduled for broadcast, or data packets of the data). The communication devices 3102 or the computing system 3100 can include one or more hearables 102. The device data 3104 or other device content can include configuration settings of the device, media content stored on the device, and/or information associated with a user of the device. Media content stored on the computing system 3100 can include any type of audio, video, and/or image data. The computing system 3100 includes one or more data inputs 3106 via which any type of data, media content, and/or inputs can be received, such as human utterances, user-selectable inputs (explicit or implicit), messages, music, television media content, recorded video content, and any other type of audio, video, and/or image data received from any content and/or data source.

The computing system 3100 also includes communication interfaces 3108, which can be implemented as any one or more of a serial and/or parallel interface, a wireless interface, any type of network interface, a modem, and as any other type of communication interface. The communication interfaces 3108 provide a connection and/or communication links between the computing system 3100 and a communication network by which other electronic, computing, and communication devices communicate data with the computing system 3100.

The computing system 3100 includes one or more processors 3110 (e.g., any of microprocessors, controllers, and the like), which process various computer-executable instructions to control the operation of the computing system 3100. Alternatively or in addition, the computing system 3100 can be implemented with any one or combination of hardware, firmware, or fixed logic circuitry that is implemented in connection with processing and control circuits which are generally identified at 3112. Although not shown, the computing system 3100 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The computing system 3100 also includes a computer-readable medium 3114, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. The disk storage device may be implemented as any type of magnetic or optical storage device, such as a hard disk drive, a recordable and/or rewriteable compact disc (CD), any type of a digital versatile disc (DVD), and the like. The computing system 3100 can also include a mass storage medium device (storage medium) 3116.

The computer-readable medium 3114 provides data storage mechanisms to store the device data 3104, as well as various device applications 3118 and any other types of information and/or data related to operational aspects of the computing system 3100. For example, an operating system 3120 can be maintained as a computer application with the computer-readable medium 3114 and executed on the processors 3110. The device applications 3118 may include a device manager, such as any form of a control application, software application, signal-processing and control module, code that is native to a particular device, a hardware abstraction layer for a particular device, and so on.

The device applications 3118 also include any system components, engines, or managers to implement interdependent human behavior detection and/or classification using active acoustic sensing. In this example, the device applications 3118 include the pre-processing module 518, the measurement module 520, and optionally the calibration module 522. Although not explicitly shown, the device applications 3118 can also include the application 406.

Throughout this disclosure, examples are described where a computing system 3100 (e.g., the hearable 102, the computing device 104, a client device, a server device, a computer, or another type of computing system) may analyze information (e.g., various audible and/or ultrasound signals) associated with a user, for example, a human behavior. Further to the descriptions above, a user 106 may be provided with controls allowing the user 106 to make an election as to both if and when systems, programs, and/or features described herein may enable collection of information (e.g., information about a user's social network, social actions, social activities, profession, a user's preferences, a user's current location), and if the user 106 is sent content or communications from a server. The computing system 3100 can be configured to only use the information after the computing system 3100 receives explicit permission from the user 106 to use the data. For example, in situations where the hearable 102 analyzes signals for human behavior detection and/or classification, individual users 106 may be provided with an opportunity to provide input to control whether programs or features of the computing system 3100 can collect and make use of the data. Further, individual users 106 may have constant control over what programs can or cannot do with the information.

In addition, information collected may be pre-treated in one or more ways before it is transferred, stored, or otherwise used, so that personally-identifiable information is removed. For example, before the computing system 3100 shares data with another device, a user 106's identity may be treated so that no personally identifiable information can be determined for the user 106. Thus, the user 106 may have control over whether information is collected about the user 106 and the user 106's device, and how such information, if collected, may be used by the computing system 3100 and/or a remote computing system.

### CONCLUSION

Although techniques using, and apparatuses including, interdependent detection and/or classification of human behavior using active acoustic sensing have been described in language specific to features and/or methods, it is to be understood that the subject of the appended claims is not necessarily limited to the specific features or methods described. Rather, the specific features and methods are disclosed as example implementations of interdependent detection and/or classification of human behavior using active acoustic sensing.

Some examples are provided below.

Example 1: A method comprising:
transmitting an acoustic transmit signal that propagates within at least a portion of an ear canal of a user;
receiving an acoustic receive signal, the acoustic receive signal representing a version of the acoustic transmit signal with one or more waveform characteristics modified due to the propagation within the ear canal;
detecting a human behavior based on the acoustic receive signal; and
controlling an operation of a device based on the detected human behavior.

Example 2: The method of example 1, wherein the device comprises at least one of a hearable or a computing device that is coupled to the hearable.

Example 3: The method of example 1 or 2, wherein the detecting of the human behavior comprises:
measuring at least two biometrics based on the one or more modified waveform characteristics of the acoustic receive signal; and
determining that the user is asleep based on the at least two biometrics.

Example 4: The method of example 3, wherein the controlling of the operation of the device comprises causing the device to switch from a normal mode to a sleep mode.

Example 5: The method of example 3 or 4, further comprising: classifying a stage of sleep based on the at least two biometrics.

Example 6: The method of any one of examples 3 to 5, wherein the at least two biometrics comprise at least two of the following:
a heart rate;
a respiration rate;
blood pressure;
occurrence or absence of muscle movement;
occurrence or absence of bruxism; and
occurrence or absence of coughing.

Example 7: The method of any previous example, wherein:
the detecting of the human behavior comprises detecting a chewing-type action performed by the user based on the one or more modified waveform characteristics of the acoustic receive signal; and
the controlling of the operation comprises causing the device to log a time associated with the chewing-type action.

Example 8: The method of any previous example, wherein:
the detecting of the human behavior comprises detecting bruxism based on the one or more modified waveform characteristics of the acoustic receive signal; and
the controlling of the operation comprises causing the device to sound an alarm responsive to the detection.

Example 9: The method of example 8, further comprising:
determining a type of bruxism based on the one or more modified waveform characteristics of the acoustic receive signal; and
causing the device to log the determined type of bruxism.

Example 10: The method of example 9, wherein the type of bruxism comprises at least one of the following:
clenching;
tapping;
side-to-side grinding; or
front-to-back grinding.

Example 11: A method comprising:
transmitting an acoustic transmit signal that propagates within at least a portion of an ear canal of a user;
receiving an acoustic receive signal, the acoustic receive signal representing a version of the acoustic transmit signal with one or more characteristics modified due to the propagation within the ear canal;
detecting a first human behavior based on the acoustic receive signal;
detecting a second human behavior based on the acoustic receive signal and the detected first human behavior, the second human behavior being different than the first human behavior; and
controlling an operation of a device based on the detected second human behavior.

Example 12: The method of example 11, wherein the device comprises at least one of a hearable or a computing device that is coupled to the hearable.

Example 13: The method of example 11 or 12, wherein the first human behavior and the second human behavior comprise different human behaviors from the following list of behaviors:
chewing;
bruxism; or
sleeping.

Example 14: The method of example 13, wherein:
the detecting of the first human behavior comprises determining that the user is sleeping based on the acoustic receive signal; and
the detecting of the second human behavior comprises detecting the bruxism based on the acoustic receive signal and the determination that the user is sleeping.

Example 15: The method of any one of examples 11-14, further comprising:
detecting an absence of a third human behavior based on the acoustic receive signal and the detected first human behavior, the third human behavior being different than the first human behavior and the second human behavior.

Example 16: The method of example 15, wherein the detecting of the absence of the third human behavior comprises determining that the user is not chewing based on the acoustic receive signal and the detected first human behavior.

Example 17: The method of any one of examples 11-16, further comprising:
transmitting another acoustic transmit signal that propagates within at least the portion of the ear canal of the user;
receiving another acoustic receive signal, the other acoustic receive signal representing a version of the other acoustic transmit signal with one or more characteristics modified due to the propagation within the ear canal;
detecting an absence of the first human behavior based on the other acoustic receive signal;
detecting a fourth human behavior based on the acoustic receive signal and the detected absence of the first human behavior, the fourth human behavior being different than the first human behavior and the second human behavior; and
controlling the operation of the device based on the fourth human behavior.

Example 18: The method of any one of examples 11-17, further comprising:
classifying the second human behavior based on the acoustic receive signal and the detected first human behavior.

Example 19: The method of example 18, wherein:
the detecting of the first human behavior comprises detecting bruxism;
the detecting of the second human behavior comprises determining that the user is sleeping; and
the classifying of the second human behavior comprises classifying a stage of sleep based on the bruxism.

Example 20: The method of example 19, further comprising:
measuring at least two biometrics based on the one or more modified characteristics of the acoustic receive signal,
wherein the classifying of the stage of sleep comprises classifying the stage of sleep based on the bruxism and the at least two biometrics.

Example 21: A computer-readable storage medium comprising instructions that, responsive to execution by a at least one processor, cause a device to perform any one of the methods of examples 1 to 20.

Example 22: A device comprising:
at least one transducer; and
at least one processor, the device configured to perform, using the at least one transducer and the at least one processor, any one of the methods of examples 1 to 20.

Example 23: The device of example 22, further comprising:
a speaker; and
an active-noise-cancellation circuit comprising a feedback microphone, wherein:
the at least one transducer comprises the speaker and the feedback microphone.

Example 24: The device of example 23, wherein the speaker and the feedback microphone are configured to be positioned proximate to one ear of a user.

Example 25: The device of example 22, wherein:
the at least one transducer comprises a speaker and a microphone;
the speaker is configured to be positioned proximate to a first ear of a user; and
the microphone is configured to be positioned proximate to a second ear of the user.

Example 26: The device of any one of examples 22 to 25, wherein the device comprises:
at least one earbud; or
headphones.

1. A method comprising:
transmitting, via a hearable of a user, an acoustic transmit signal that propagates within at least a portion of an ear canal of the user;
receiving an acoustic receive signal, the acoustic receive signal representing a version of the acoustic transmit signal with one or more of an amplitude, a phase, or a frequency modified due to the propagation within the ear canal;
detecting a first human behavior based on the acoustic receive signal;
detecting a second human behavior based on the acoustic receive signal and the detection of the first human behavior, the second human behavior being different than the first human behavior, the first and second human behaviors comprising at least one of chewing, eating, bruxism, or sleeping; and
controlling an operation of a device based on the detection of the second human behavior.

2. The method of claim 1, wherein the device comprises at least one of the hearable or a computing device that is coupled to the hearable.

3. The method of claim 1, wherein detecting sleeping comprises monitoring or measuring one or more biometrics of the user.

4. The method of claim 3, wherein:
the detecting of the first human behavior comprises determining that the user is sleeping based on the acoustic receive signal; and
the detecting of the second human behavior comprises detecting the bruxism based on the acoustic receive signal and the determination that the user is sleeping.

5. The method of claim 1, further comprising:
detecting an absence of a third human behavior based on the acoustic receive signal and the detected first human behavior, the third human behavior being different than the first human behavior and the second human behavior.

6. The method of claim 5, wherein the detecting of the absence of the third human behavior comprises determining that the user is not chewing based on the acoustic receive signal and the detected first human behavior.

7. The method of claim 1, further comprising:
transmitting another acoustic transmit signal that propagates within at least the portion of the ear canal of the user;
receiving another acoustic receive signal, the other acoustic receive signal representing a version of the other acoustic transmit signal with one or more characteristics modified due to the propagation within the ear canal;
detecting an absence of the first human behavior based on the other acoustic receive signal;
detecting a fourth human behavior based on the other acoustic receive signal and the detected absence of the first human behavior, the fourth human behavior being different than the first human behavior and the second human behavior; and
controlling the operation of the device based on the fourth human behavior.

8. The method of claim 1, further comprising:
classifying the second human behavior based on the acoustic receive signal and the detected first human behavior.

9. The method of claim 8, wherein:
the detecting of the first human behavior comprises detecting bruxism;
the detecting of the second human behavior comprises determining that the user is sleeping; and
the classifying of the second human behavior comprises classifying a stage of sleep based on the bruxism.

10. The method of claim 9, further comprising:
measuring at least two biometrics based on the one or more modified characteristics of the acoustic receive signal,
wherein the classifying of the stage of sleep comprises classifying the stage of sleep based on the bruxism and the at least two biometrics.

11. A non-transitory computer-readable storage medium comprising instructions that, responsive to execution by a processor, is configured to:
detect a first human behavior based on an acoustic receive signal, the acoustic receive signal representing a version of an acoustic transmit signal with one or more of an amplitude, a phase, or a frequency modified due to propagation of the acoustic transmit signal within an ear canal of a human;
detect, via the processor, a second human behavior based on the acoustic receive signal and the detection of the first human behavior, the second human behavior being different than the first human behavior, the first and second human behaviors comprising at least one of chewing, eating, bruxism, or sleeping; and
control, via the processor, an operation of a device based on the detection of the second human behavior.

12. A device comprising:
a hearable comprising at least one transducer configured to:
transmit an acoustic transmit signal that propagates within at least a portion of an ear canal of the user; and
receive an acoustic receive signal, the acoustic receive signal representing a version of the acoustic transmit signal with one or more of an amplitude, a phase, or a frequency modified due to the propagation within the ear canal; and
at least one processor configured to:
detect a first human behavior based on the acoustic receive signal;
detect a second human behavior based on the acoustic receive signal and the detection of the first human behavior, the second human behavior being different than the first human behavior, the first and second human behaviors comprising at least one of chewing, eating, bruxism, or sleeping; and
control an operation of the device based on the detection of the second human behavior.

13. The device of claim 12, further comprising:
a speaker; and
an active-noise-cancellation circuit comprising a feedback microphone, wherein:
the at least one transducer comprises the speaker and the feedback microphone.

14. The device of claim 13, wherein the speaker and the feedback microphone are configured to be positioned proximate to one ear of the user.

15. The device of claim 12, wherein:
the at least one transducer comprises a speaker and a microphone;
the speaker is configured to be positioned proximate to a first ear of a user; and
the microphone is configured to be positioned proximate to a second ear of the user.

16. The device of claim 12, wherein the device comprises at least one earbud.

17. The non-transitory computer-readable storage medium of claim 11, wherein:
the first human behavior comprises bruxism;
the second human behavior comprises sleeping; and
the non-transitory computer-readable storage medium is further configured to classify a stage of sleep based on the bruxism.

18. The non-transitory computer-readable storage medium of claim 11, wherein:
the first human behavior comprises sleeping; and
the second human behavior comprises bruxism.

19. The non-transitory computer-readable storage medium of claim 18, wherein the non-transitory computer-readable storage medium is further configured to:
detect an absence of a third human behavior based on the acoustic receive signal and the detected first human behavior, the third human behavior being different than the first human behavior and the second human behavior.

20. The non-transitory computer-readable storage medium of claim 19, wherein the third human behavior comprises chewing.
